(19) United States
(12) Patent Application Publication
(10) Pub. No.: US 2018/0368883 A1
(43) Pub. Date: Dec. 27, 2018

(54) HAND-HELD DEVICE AND COMPUTER-IMPLEMENTED SYSTEM AND METHOD FOR ASSISTED STEERING OF A PERCUTANEOUSLY INSERTED NEEDLE

(71) Applicant: THE GOVERNORS OF THE UNIVERSITY OF ALBERTA, Edmonton (CA)

(72) Inventors: Carlos Rossa, Edmonton (CA); Ron Sloboda, St. Albert (CA); Nawaid Usmani, Edmonton (CA); Mahdi Tavakoli, Edmonton (CA)

(73) Assignee: THE GOVERNORS OF THE UNIVERSITY OF ALBERTA, Edmonton (CA)

(21) Appl. No.: 15/978,996

(22) Filed: May 14, 2018

Related U.S. Application Data
(60) Provisional application No. 62/507,612, filed on May 17, 2017.

(51) Int. Cl. A61B 17/34; A61B 17/32; G06T 7/70; G06T 7/20

(52) U.S. Cl. CPC A61B 17/3403; A61B 17/320068; G06T 7/70; A61B 2017/0042; A61B 17/3468; G06T 2207/10132; A61B 2562/0219; G06T 7/20

(57) ABSTRACT

A hand-held device for assisted steering of a percutaneously inserted needle comprises a handle, an actuation unit, and a haptic feedback unit. A computer-implemented system calculates a needle shape and position based on one or a combination of analysis of ultrasound images and determination of needle insertion parameters based on electronic signals generated by a sensor unit. The system calculates a correction to a needle insertion parameter to achieve a target needle trajectory, including a correction to a needle axial rotation. The system activates the actuation unit to rotate the needle in accordance with the correction to the needle axial rotation, and activates to the haptic feedback unit to vibrate the handle in a vibration pattern determined by a rules database depending on one or a combination of the calculated needle shape, the calculated needle position, and the correction to the needle insertion parameter.

### CROSS REFERENCE TO RELATED APPLICATION

This application claims the benefit of U.S. Provisional Patent Application Ser. No. 62/507,612, filed May 17, 2017, which is hereby incorporated by reference in its entirety.

### TECHNICAL FIELD

The present invention relates to a hand-held device and computer-implemented systems and methods for real-time assisted steering of percutaneously inserted needles, such as may be used during prostate brachytherapy.

### BACKGROUND OF THE INVENTION

Prostate brachytherapy is an effective treatment for prostate cancer due to its excellent success rates, favorable toxicity profile, and non-invasiveness. In conventional prostate brachytherapy, a surgeon inserts a long, flexible needle loaded with radioactive seeds through the perineum into the patient's body. With the guidance of a grid template and ultrasound images generated by a transrectal probe, the surgeon manually steers the needle toward preplanned locations in the prostate gland and then deposits the radioactive seeds. The radioactive seeds emit radiation to kill the tumor cells in a short vicinity of the treated area, while minimizing radiation exposure to adjacent critical structures.

Accurate radioactive seed placement is critical to effective prostate brachytherapy. In practice, however, the needle may not travel on the planned straight path, resulting in deviations of actual radioactive seed placement from the planned locations. Factors contributing to such imprecision include prostate gland tissue deformation and/or needle deflection. In regard to the latter factor, brachytherapy needles typically have a beveled tip to facilitate cutting through patient tissue. However, asymmetrical forces act on the beveled needle tip, causing it to deflect from the planned straight path. Previous work has shown that seeds can be placed with an average absolute accuracy of 5 mm, which is more than 10% of the average prostate gland diameter [4]. This substantial error narrows the scope of brachytherapy to primarily treating the entire prostate gland for patients with localized prostate cancer.

To address the need for needle targeting accuracy, fully automated robotic systems have been developed to automatically insert a needle and control its trajectory towards target locations in the prostate (see [5] to [12]). To create the steering effect, these systems rotate the needle base to change the orientation of the beveled tip and consequently the direction of the resultant force is then used to control the direction of needle deflection. However, to date, no such system has been deployed in clinical practice due to complexities and significant modifications that would be necessary in the procedure and the operating room.

### SUMMARY OF THE INVENTION

The present invention provides real-time assistance to a surgeon to precisely, efficiently, and intuitively position a needle percutaneously inserted in a patient to achieve a target needle trajectory, while allowing the surgeon to maintain at least partial manual control over needle insertion.

It will be understood that the present invention is used with a needle extending axially between a proximal end and a distal end comprising a beveled needle tip.

In one aspect, the present invention comprises a hand-held device for assisted steering of a percutaneously inserted needle. In embodiments, the device comprises:
(a) a handle for manual gripping of the device by a user of the device;
(b) an actuation unit attached to the handle, the actuation unit comprising:
   (i) a rotary actuator for axially rotating the needle relative to the handle;
   (ii) an axial actuator for inducing axial micro-vibrations of the needle relative to the handle;
   wherein the rotary actuator and the axial actuator are simultaneously operable to simultaneously axial rotate the needle relative to the handle and induce axial micro-vibrations of the needle relative to the handle; and
(c) a haptic feedback unit for inducing vibrations in the handle.

In embodiments of the device, the device may further comprise a sensor unit comprising at least one sensor attached to the handle for generating, in response to movement of the device, an electronic signal indicative of a needle insertion parameter comprising one or a combination of the needle position, a needle orientation, a needle axial rotation angle, a needle velocity, and a needle acceleration. In embodiments of the device, the at least one sensor may comprise one or a combination of an accelerometer and a gyroscopic sensor.

In another aspect, the present invention comprises a computer-implemented system for assisted steering of a percutaneously inserted needle. In embodiments, the system comprises:
(a) a hand-held device comprising:
   (i) a handle for manual gripping of the device by a user of the device;
   (ii) an actuation unit attached to the handle, the actuation unit comprising:
      (1) a rotary actuator for axially rotating the needle relative to the handle;
      (2) an axial actuator for inducing axial micro-vibrations of the needle relative to the handle,
      wherein the rotary actuator and the axial actuator are simultaneously operable to simultaneously axial rotate the needle relative to the handle and induce axial micro-vibrations of the needle relative to the handle;
   (iii) a haptic feedback unit for inducing vibrations in the handle;
(b) a sensor unit comprising at least one sensor for generating, in response to movement of the device, an electronic signal indicative of a needle insertion parameter comprising one or a combination of the needle position, a needle orientation, a needle axial rotation angle, a needle velocity, and a needle acceleration;
(c) a display device; and
(d) a computer operatively connected to the device and the display device, the computer comprising a processor and a memory comprising a non-transitory computer readable medium storing instructions executable by the processor to implement, in real-time, with the insertion of the needle, a method comprising the steps of:
   (i) determining a location of a portion of the needle;
   (ii) calculating a needle insertion parameter comprising one or a combination of a needle position, a needle orientation, a needle axial rotation angle, a needle velocity, and a needle acceleration, wherein the calculating is based on an electronic signal from the sensor unit;
   (iii) calculating a needle shape and a needle position, wherein the calculating is based on one or a combination of the determined location of the portion of the needle and the calculated needle insertion parameter;
   (iv) displaying on the display device one or a combination of the calculated needle shape, the calculated needle position, and the calculated needle insertion parameter;
   (v) calculating a correction to the needle insertion parameter for a target needle trajectory, wherein the calculating is based on one or a combination of the calculated needle shape, the calculated needle position, and the calculated needle insertion parameter, and wherein the correction to needle insertion parameter comprises at least either a correction to the needle axial rotation angle or a needle rotation depth paired with a discrete needle axial rotation angle;
   (vi) controlling the rotary actuator of the device to axially rotate the needle by either the correction to the needle axial rotation angle or by the discrete needle axial rotation angle at the paired needle rotation depth;
   (vii) activating the haptic feedback unit of the device to vibrate the handle of the device in a vibration pattern, wherein the vibration pattern is determined by a rules database depending on one or a combination of the calculated needle shape, the calculated needle position, and the calculated correction to the needle insertion parameter; and
   (viii) repeating steps (i) to (vi).

In embodiments of the system, the sensor of the sensor unit is attached to the handle and comprises at least one or a combination of an accelerometer and a gyroscopic sensor.

In embodiments of the system, the sensor unit comprises a camera for tracking the position of the device.

In embodiments of the system, the step of determining the location of the portion of the needle comprises processing an ultrasound image of the needle.

### BRIEF DESCRIPTION OF DRAWINGS

In the drawings, like elements are assigned like reference numerals. The drawings are not necessarily to scale, with the emphasis instead placed upon the principles of the present invention. Additionally, each of the embodiments depicted is but one of a number of possible arrangements utilizing the fundamental concepts of the present invention. The drawings are briefly described as follows:

FIGS. 1A and 1B are a distal perspective view and a proximate perspective view, respectively, of an embodiment of a hand-held device of the present invention.

FIG. 2 is a side cross-sectional view of another embodiment of a hand-held device of the present invention, similar to the device shown in FIGS. 1A and 1B.

FIGS. 3A and 3B are an assembled perspective view and an exploded perspective view, respectively, of an actuation unit of the device shown in FIG. 2.

FIG. 4 (PRIOR ART) is a depiction of a cantilever compliant beam model of a needle used to predict needle deflection in a needle-tissue interaction model.

FIGS. 5A-F is a series of graphs showing: (FIG. 5A) control variable u(d) as a function of the needle rotation depth dg; (FIG. 5B) and (FIG. 5C) the cost functions J penalizing needle targeting error for Case 1 and Case 2, respectively; (FIG. 5D), (FIG. 5E) and (FIG. 5F) hypothetical examples of candidate rotation points within the control horizon [d, dₕ] and the resultant deflection in [d, d_{f}].

FIGS. 6A-D is a series of graphs showing: (FIG. 6A) and (FIG. 6B) the optimal rotation depth for Case 1 for d_{f}=130 and d_{f}=150 mm, respectively; (FIG. 6C) and (FIG. 6D), the depth of first (d₁) and second (d₂) rotation, respectively, that minimizes the cost function for Case 2 for df=150 mm.

FIGS. 7A-D show an overview of the needle steering controller, with FIG. 7A showing the block diagram of the needle steering system; in FIG. 7B, the RRT algorithm evaluates the needle targeting accuracy for different rotation depths as shown in FIG. 7C; in FIG. 7D, the resultant set of rotation depths.

FIG. 8A is a schematic block diagram of an embodiment of the computer-implemented system of the present invention, in relation to a surgeon, a patient, a needle, and a transrectal ultrasound probe.

FIG. 8B shows a schematic depiction of a system for determination of needle shape and position using a partial sagittal ultrasound image observation, and FIG. 8C shows experimental results for two different phantom tissues.

FIG. 8D shows a schematic depiction of system for determination of needle shape and position using a series of transverse ultrasound images; FIG. 8E needle shape estimation results; and FIG. 8F needle tip tracking experimental results.

FIG. 9 depicts an experimental set up for testing an embodiment of a prototype of the computer-implemented system of the present invention.

FIG. 10 is a schematic block diagram of the embodiment of the prototype system of FIG. 9.

FIG. 11 is a graph showing measured needle-tissue axial insertion forces for different vibration frequencies applied to the needle shaft in experiments conducted with the prototype system of FIG. 9.

FIG. 12 is a series of graphs showing for experiments with the prototype system of FIG. 9 with different tissue samples: (top, first panel) the measured needle tip deflection and the predicted needle tip deflection using the identified model parameters; (middle, second panel) the error between the predicted and measured needle tip deflection; and (bottom, third panel) the observed tip force.

FIG. 13 shows model fit results for each tissue sample. The model parameters are found by minimizing the difference between the measured and estimated needle tip deflection at the depth of 140 mm.

FIGS. 14A and 14B shows the path followed by the needle tip in the X and Y planes (defined in FIG. 9) during insertion in porcine, bovine, and synthetic tissue and the average position of the bevel angle using open loop (FIG. 14A) and closed loop (FIG. 14B) controllers, for each of the 15 insertions. Only the deflection in the X is controlled.

FIG. 15A shows the seed tracking routine in ultrasound images. FIG. 15B shows the image processing. Ultrasound images captured during a Phase 3 showing the last implanted seed to be localized, with the tracking algorithm steps shown underneath.

FIGS. 16A and 16B shows experimental results of seed deposition following a hypothetical pre-planning using open loop needle steering (FIG. 16A) and closed loop needle steering (FIG. 16B). The solid dot indicates the seed target location. The open circle is the position of the needle tip at the target depth, and the open square shows the final position of the centroid of each seed after the needle is withdrawn.

FIG. 17 shows dummy seed displacement from the deposition location during needle withdrawal in each tissue with open loop (left) and closed loop (right) needle steering controllers.

### DETAILED DESCRIPTION

The present invention relates to a hand-held device for real-time assisted steering a percutaneously inserted needle, and related computer-implemented systems and methods. Any term or expression not expressly defined herein shall have its commonly accepted definition understood by a person skilled in the art. As used herein, the “real time” in describing a series of steps means that the steps are completed within a time period that is, in embodiments, within about 0.1 seconds, 0.2 seconds, 0.5 seconds, 1 second, or 5 seconds or seconds.

### Needle and Stylet

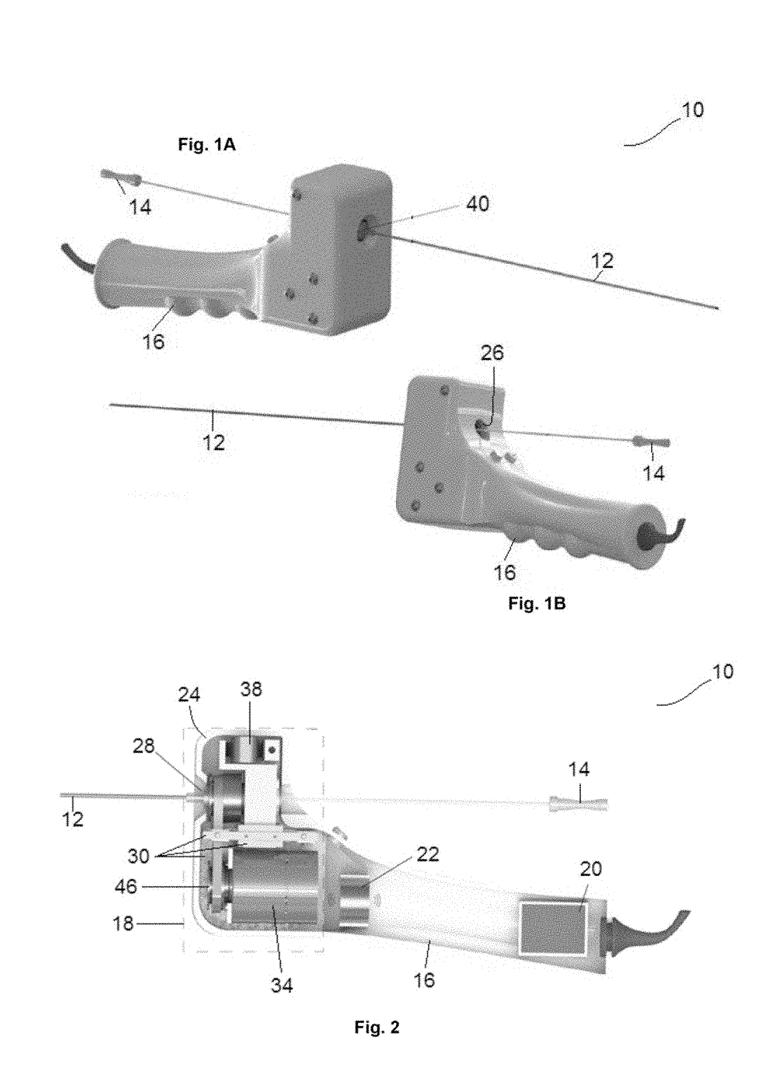

Referring to FIGS. 1A and 1B, it will be understood that the device 10 of the present invention may be used with an elongate conventional brachytherapy needle 12 with a needle shaft having a needle hub, and extending axially between a proximal end and a distal end comprising a beveled needle tip. In an exemplary embodiment, the needle 12 may be a standard 18-gauge hollow brachytherapy needle. The needle 12 is loaded with radioactive seeds in the needle lumen, and a stylet 14 inserted into the needle lumen. As is known to those skilled in the art, a surgeon pushes the device 10 towards a patient to insert the needle 12 until a target depth is reached, whereupon the surgeon retains the stylet 14 in place while retracting the device 10 and the needle 12 so that the stylet 14 pushes the radioactive seeds from inside the needle 12 out of the needle lumen and deposits them into the prostate gland. As the beveled needle tip cuts through tissue, tissue displacement at the edge of the bevel needle tip creates a resultant force normal to the needle shaft that causes it to bend on a curved trajectory. Hence, changing the orientation of the beveled needle tip by axial rotation of the needle base changes the direction of the force applied at the beveled needle tip, causing the needle 12 to bend in a different direction. Thus, a proper combination of needle translation and axial rotation can force the needle tip to follow a desired trajectory [15, 16].

### Hand-Held Device

FIGS. 1A, 1B and 2 show an exemplary hand-held device 10 of the present invention with a conventional needle 12 and stylet 14. In an exemplary embodiment, the device 10 is constructed so as to weigh less than about 0.16 kg (0.35 lb.), facilitating its use for conventional needle insertion techniques as described above. In the exemplary embodiment, the device 10 comprises a handle 16 with an attached actuation unit 18, sensor unit 20, and haptic feedback unit 22, as are described below. In other exemplary embodiments, the sensor unit 20 may be partially or wholly detached from the hand-held device 10.

### Handle

A purpose of the handle 16 is to provide a member that may be manually gripped by a surgeon so as to allow the surgeon to maintain manual control over the needle insertion depth. In the exemplary embodiment shown in FIGS. 1A, 1B and 2, the handle 16 is a substantially cylindrical member having a length of approximately 140 millimeters, and a diameter of approximately 30 millimeters. The handle 16 may be contoured with ridges that engage the surgeon's fingers so as to prevent slipping of the device in the surgeon's hands. The handle 16 may also be provided with control knobs. In the exemplary embodiment, the handle 16 is 3D-printed from plastic and extends into a monolithically printed housing 24 that encloses the actuation unit 18, haptic feedback unit 22 and sensor unit 20, thereby attaching these components to the handle 16. The housing 24 defines an aperture 26 that allows for through passage of the needle 12 and the stylet 14. In other exemplary embodiments (not shown), the handle 16 may have a different configuration and be made of other materials, and manufactured using different processes, known in the art.

### Actuation Unit

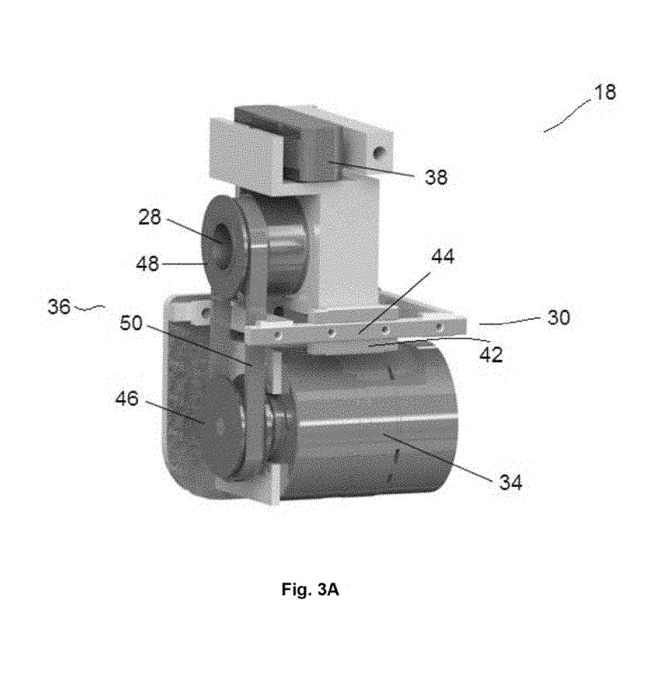

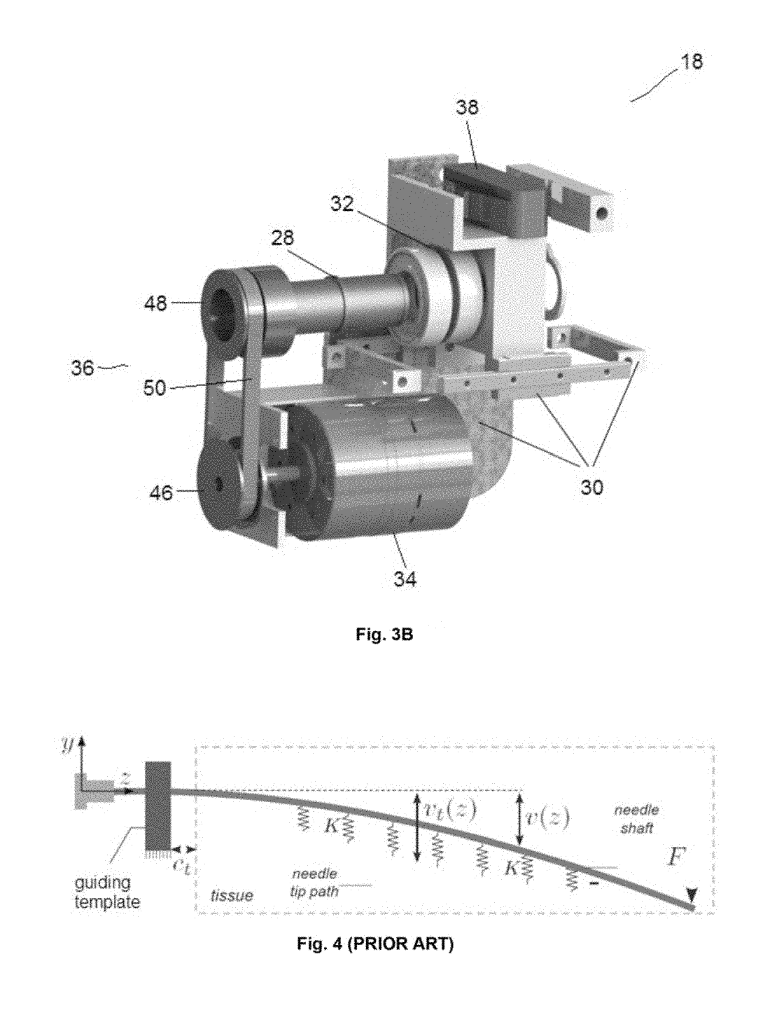

In the exemplary embodiment shown in FIGS. 3A and 3B, the actuation unit 18 comprises a needle holder 28, support structure 30, a rotary bearing assembly 32, a rotary actuator 34, a rotary encoder, drivetrain 36, and an axial actuator 38, all of which are operatively connected to an electrical power source such as an electric power cord or an electrochemical battery. It will be appreciated that the configuration of the actuation unit 18 allows for simultaneous operation of the rotary actuator 34 and the axial actuator 38 so as to simultaneously rotate the needle 12 about its axis, and to vibrate the needle 12 in the axial direction.

### Needle Holder

A purpose of the needle holder 28 is to attach the needle 12 to the other components of the actuation unit 18. In the exemplary embodiment shown in FIGS. 3A and 3B, the needle holder 28 comprises a threaded tubular member that extends through the aperture 26 of the housing 24. In use, the needle 12 is inserted through the needle holder 28 so that the needle hub 40 is placed within the needle holder 28, whereupon the needle holder 28 may be secured to the needle 12 by a quick-snap (and quick-release) mechanism.

### Support Structure

A purpose of the support structure 30 is to provide a member or members for attachment of the actuation unit 18 directly or indirectly to the handle 16. In the exemplary embodiment, the support structure 30 comprises a pair of plates 42 and plurality of linear rails 44. The outward facing surfaces of the plates 42 attach to the inside of the housing 24. It will be understood that in FIGS. 3A and 3B, one of the plates 42 has been removed to show the components of the actuation unit 18. The inward facing surfaces of the plates 42 attach to miniature linear rails 44, which in turn provide mounting points for the other components of the device 10. The needle holder 28 and housing assembly 24 slide on two of miniature linear rails 44 such that they can translate axially—that is, in the direction of needle insertion.

### Rotary Bearing Assembly

A purpose of the rotary bearing assembly 32 is to permit axial rotation of the needle holder 28 (and hence the needle 12) relative to the handle 16. In the exemplary embodiment, the rotary bearing assembly 32 comprises an outer race, an inner race, and bearing elements (concealed from view). The outer race is secured to the support structure 30. The inner race is secured circumferentially around the needle holder 28. The bearing elements permit the inner race (and hence the needle holder 28) to rotate axially relative to the outer race (and hence the support structure 30 and handle 16).

### Rotary Actuator

A purpose of the rotary actuator 34 is to axially rotate the needle 12 in a controlled manner. In the exemplary embodiment, the rotatory actuator 34 comprises an electric motor, a drivetrain 36, and a rotary encoder. A purpose of the electric motor is to convert electrical energy from the electrical power source to rotation of a motor rotor. In an exemplary embodiment, the motor is a DC motor model 26195024SR from Faulhaber™ (Croglio, Switzerland), having embedded reduction gears with a 33:1 reduction ratio, and powered by a L298N PMW drive.

### Drivetrain

A purpose of the drivetrain 36 is to transmit rotation of the motor rotor to axial rotation of the needle holder 28. In the exemplary embodiment, the drivetrain 36 comprises a first pulley 46, a second pulley 48, and a belt 50. The first pulley 46 is attached to the motor rotor for rotation therewith. The second pulley 48 is attached to the needle holder 28. The belt 50 is looped over the first pulley 46 and the second pulley 48 to transmit rotation of the first pulley 46 to the second pulley 48. The distance separating the needle 12 and the motor shafts plus half of the circumference of each pulley 46, 48 gives the length of the belt 50 around the pulleys 46, 48. In order to allow for simultaneous needle rotation and some small longitudinal relative translation of the pulleys 46, 48, a 2 mm clearance is added to the length of the belt 50.

### Rotary Encoder

A purpose of the rotary encoder is to generate electronic signals that are indicative of the angular rotational position of the motor rotor (and hence the angular rotation position of the needle holder 28 and the needle 12). Rotary encoders are electromechanical devices that are well known to persons skilled in the art. A variety of different types of rotary encoders may be suitable for use with the device 10. In the exemplary embodiment, the encoder is an incremental encoder with 16 pulses per revolution connected to the gear of the electric model that permits the angular position of the needle shaft to be measured with 0.1 degree accuracy. In other embodiments, electromechanical means other than a rotary encoder may be used to measure the angular rotational position of the needle 12 by detecting the position of the motor drive shaft or the needle holder 28. Such devices may include with limitation, a synchro transducer, an electrical resolver transformer, a rotary variable differential transformer, or potentiometers.

### Axial Actuator

A purpose of the axial actuator 38 is to induce high-frequency, axial micro-vibrations in the needle 12. As used herein, the term “micro-vibrations” refers to oscillations that are less than or equal to about 0.04 millimeters in amplitude. The reason for inducing micro-vibrations is that translational friction along the needle shaft can be reduced by modulating a vibratory low-amplitude displacement onto a regular needle insertion profile [17]. This can make the needle insertion easier for the surgeon and potentially reduce tissue deformation. In addition, the micro-vibrations can allow for easy detection of the needle tip under Doppler ultrasound imaging [18]. The axial actuator 38 may comprise any electro-mechanical transducer that is suitable for converting electrical signals to oscillating movements that will generate axial micro-vibrations in the needle holder 28. In an exemplary embodiment, the axial actuator 38 is an amplified piezoelectric actuator (APA60S from Cedrat Technologies™, Meylan, France), powered by a piezo-electric drive (PDm200, PiezoDrive™, Callaghan, Australia).

### Sensor Unit

A purpose of the sensor unit 20 is to generate electronic signals in real-time that are indicative of, or may be used to derive, the position, velocity, acceleration and orientation of the device 10. In an exemplary embodiment, the sensor unit 20 is attached to the device 10 and may comprise one or more types of sensors for measuring the position or motion of the device 10, including without limitation inertial sensing technologies such as accelerometers and gyroscopes. Accelerometers and gyroscopic sensors are well known to persons skilled in the art. A variety of different types of accelerometers and gyroscopic sensors may be suitable for use with the device 10, including without limitation micro-electronical systems (MEMS)-based accelerometers and gyroscopic sensors.

In other embodiments, the sensor unit 20 may be at least partially detached from the hand-held device 10. For instance, in the exemplary embodiment of the prototype system described in Example 1, the sensor unit 20 implements optical tracking technology wherein the sensor unit 20 comprises cameras that detect the movement of tracking markers attached to the handle 16. Optical sensing technology is well known to person skilled in the art.

In other embodiments, the sensor unit 20 may be embedded within the hand-held device 10. For instance, in the exemplary embodiment of the prototype system described in Example 2, a compression/traction sensor is embedded in the hand-held device 10 to measure the axial force applied to the needle base during insertion and withdrawal (model LSB200 S-Beam from Futek, Irvine, USA). The force measurements from two 1-DOF force sensors during needle insertion and withdrawal may be used to estimate the forces applied by the tissue onto the needle tip, such that future needle deflection can be predicted by a mechanics-based model and the necessary corrective action taken by the hand-held device 10.

### Haptic Feedback Unit

A purpose of the haptic feedback unit 22 is to induce vibrations in the handle 16 so as to provide the surgeon with tactile alerts of the need or lack of need for corrective maneuvering of the device 10. The haptic feedback unit 22 may comprise any electro-mechanical transducer that is suitable for converting electrical signals to oscillating movements that will induce vibrations in the handle 16 that can be sensed by the surgeon. For example, electro-mechanical transducers used in smart phones to create vibration alerts may be suitable for use in the haptic feedback unit 22.

### Needle—Tissue Interaction Model

An object of the computer-implemented system and method of the present invention is to determine control commands to be applied to the device so that the needle moves along a target needle trajectory. This requires a needle-tissue interaction model to predict needle deflection. It will be appreciated that needle-tissue interaction models other than the particular models 1 and 2 described below may be suitable for use with the present invention so long as the model allows the calculation of the deflected shape of the needle. In the exemplary embodiment, the objective is to develop a model that can be entirely identified using only 2D ultrasound images of the needle in tissue, which are often available in clinical settings. In prostate brachytherapy, the needle ideally follows a straight line trajectory. Hence, and as there is no need to generate 3D trajectories, the model may be limited to planar needle deflections.

i. Needle—Tissue Interaction Model 1

In order to predict needle deflection during insertion, the needle is modelled as a cantilever compliant beam that undergoes forces applied by the tissue as shown in FIG. 4. According to the Galerkin-Bubnov method [19], beam deflection can be approximated as the sum of n candidate shape functions (eigenfunctions), each of which represents a mode of vibration. The deflection v(dz) of a needle at a point z along its shaft and, for a given insertion depth d in this case, can be defined as

$$v ( d , z ) = {\sum }_{i = 0}^{n} {q}_{i} ( z ) {g}_{i} ( d ) \qquad ( 1 )$$

where qᵢ(z) is the displacement of the needle (deflection) at each point z along its shaft and gᵢ(d) is a weighting coefficient (eignenvalue) for each of the n assumed vibration modes. The eingenfunctions qᵢ(z) must satisfy the boundary conditions of a cantilever beam and be differentiable at least up to the highest order of the partial differential equations of the beam. For a cantilever beam of length L, the deflection can be given by [19]

$$\begin{matrix}{q}_{i} ( z ) = \frac{1}{{κ}_{i}} [ sin ξ ( z ) - sinh ξ ( z ) - {γ}_{i} \{ cos ξ ( z ) - cosh ξ ( z ) \} ] \\ where & ( 2 ) \\ ξ ( z ) = {β}_{i} \frac{z}{L} & ( 3 )\end{matrix}$$

and the constants γᵢ and κᵢ are computed as

$${γ}_{i} = \frac{sin {β}_{i} + sinh {β}_{i}}{cos {β}_{i} + cosh {β}_{i}} \\ {κ}_{i} = sin {β}_{i} - sinh {β}_{i} - {γ}_{i} ( cos {β}_{i} - cosh {β}_{i} ) . \qquad ( 4 )$$

The values of the constants pi for a clamped-free beam are β₁=1.857, β₂=4.695, β₃=7.855, β₄=10.996, and βᵢ≈π(i−½) for i>4 [19]. At this stage the assumed displacement functions are entirely parametrized. In the following, it is demonstrated that the weighting coefficients gᵢ(d) can be given as functions of the needle-tissue interaction forces such that the system reaches equilibrium.

A. Needle-Tissue Equilibrium

To calculate the weighting coefficients gᵢ(d), a variational method known as the Rayleigh-Ritz method is used in which equilibrium of the system is established using the principle of minimum potential energy. This approach has been previously employed to estimate needle deflection in [20]. In the present invention, the tissue model accounts for unlimited number of needle rotations while accounting for tissue displacement. In addition, the mathematical approach reduces the model to a simple system of linear equations, making it computationally efficient, and enabling it to be parametrized using only ultrasound images of the needle during insertion.

The coefficients gᵢ(d) must minimize the system potential Π(d) defined by

Π(d)=U(d)+V(d) (5)

where U (d) is the total stored energy in the system and V (d) is the work done by conservative forces. The expressions for the potential energy and the work for the needle-tissue system are now derived.

As said earlier, as the needle tip cuts through the tissue, the bevel creates a resultant normal force F at the needle tip (see FIG. 4). Other forces applied at the bevel will be neglected as they mostly induce axial compression of the needle. As the needle bends, the work due to F is

V(d)=−Fv(d,L). (6)

which is added to Π(d) in (5). The bending strain energy stored in the needle as a result of deflection is

$${U}_{b} ( d ) = \frac{1}{2} {\int }_{O}^{L} {EI ( \frac{{\partial }^{2} v ( d , z )}{\partial {z}^{2 }} )}^{2} dz , \qquad ( 7 )$$

where E and I are the needle Young's modulus of elasticity and its second moment of inertia, respectively.

In brachytherapy, the needles are inserted through a guiding template to help guide the needle towards a target and to minimize deflection outside tissue. The target is usually defined on a straight line from the needle location in the template to a desired depth in tissue. The template is modelled as a rigid spring of stiffness Kₚ>>0, which has no thickness. The spring is connected to the needle shaft at a distance of zₜ from the needle's base with zₜ=L−d−cₜ, where cₜ is the distance from the template to the tissue surface (see FIG. 4). The potential energy stored in the template is

Uₚ(d)=½Kₚv(d,zₜ)². (8)

As the needle bends, the shaft moves and deforms the surrounding tissue. In turn, the compressed tissue applies forces to the needle shaft. Assuming small local magnitude and deformation velocity of the tissue, it is reasonable to assume that the tissue is a purely elastic medium. Thus, the force applied to the needle at a certain point along the shaft becomes proportional to the tissue displacement at that point. If vₜ(z) is the initial position of the uncompressed tissue, the tissue reaction force is K(v(d, z)−vₜ(z)), where K is the stiffness of the tissue per unit length of the needle and vₜ(z) is the path cut by the needle tip. Therefore, the energy due to tissue compression is

Uₜ(d)=½K∫_{L-d}^{L}[v(d,z)−vₜ(d,z)]²dz (9)

As the model essentially compares the current needle shape with the path cut by the needle tip, it can automatically account for an unlimited number of needle rotations.

B. Calculating the Eigenvalues gᵢ(d)

Now that all the components of the system potential Π(d) have been defined, the weighting coefficients gᵢ(d) can be calculated using the principle of minimum potential energy. According to the Rayleigh-Ritz method [21], the coefficients gᵢ(d) must give δΠᵢ=0 for any values of δgᵢ where δ denotes infinitesimal difference. Therefore, gᵢ(d) must satisfy:

$$\frac{\partial {Π}_{i} ( d )}{\partial {g}_{i} ( d )} = \frac{\partial }{\partial {g}_{i} ( d )} ( {U}_{b} + {U}_{p} + {U}_{t} + V ) = 0 \qquad ( 10 )$$

Replacing (6)-(9) in (10) and taking the partial derivative with respect to gᵢ(d) yields

$$EI {\int }_{O}^{L} ( {\sum }_{i = 1}^{n} {\tilde{q}}_{i} ( z ) {g}_{i} ( d ) ) {\tilde{q}}_{i} ( z ) dz + {K}_{p} ( {\sum }_{i = 1}^{n} {q}_{i} ( {z}_{i} ) {g}_{i} ( d ) ) {q}_{i} ( {z}_{i} ) + K {\int }_{L - d}^{L} ( {\sum }_{i = 1}^{n} {q}_{i} ( z ) {g}_{i} ( d ) ) {q}_{i} ( z ) dz - K {\int }_{K - d}^{L} {V}_{i} ( {d}_{i} z ) {q}_{i} ( z ) dz = F , \qquad ( 11 )$$

where the double dot denotes the second derivative of qᵢ(z) with respect to z.

In order to isolate the weighting coefficients gᵢ(d) in the previous equation, four supplementary variables are created and defined as follows:

$${ψ}_{ji} = {\int }_{O}^{L} {\tilde{q}}_{i} ( z ) {\tilde{q}}_{j} ( z ) dz , {ω}_{ji} = {\int }_{L - d}^{L} {q}_{i} ( z ) {q}_{j} ( z ) dz , {γ}_{ji} = {q}_{i} ( {z}_{i} ) {q}_{j} ( {z}_{i} ) , {φ}_{i} = {\int }_{L - d}^{L} {v}_{i} ( d , z ) {q}_{i} ( z ) dz . \qquad ( 12 )$$

After some straightforward manipulation, the previous equation rearranges as

$${\sum }_{j = 1}^{n} [ {g}_{j} ( d ) ( EI {ψ}_{ji} + K {ω}_{ji} + {K}_{p} {γ}_{ji} ) ] - K {φ}_{i} = F \qquad ( 13 )$$

This equation shows that the model has been reduced to a system composed of n linear equations. This is the closed form solution through which the coefficients gᵢ(d) are found in order to calculate the needle deflection given in (1).

C. Tip Force Estimator

The proposed model requires only two input parameters, i.e., the tissue stiffness K and the force at the needle tip F. The first one can be obtained experimentally by model fitting and can be considered to be constant throughout the insertion. To identify the second parameter, an observer is developed in order to calculate the force F as the needle is inserted. To this end, it is assumed that the deflection of the needle tip can be acquired from ultrasound images of the needle in tissue, that will be referred to as v_{L}. Therefore, from (1), and knowing that qᵢ(L)=1 ¤i, it yields:

v(L,d)=g₁(d)+g₂(d)+ . . . gₙ(d)=v_{L} (14)

Now, adding this equation to the system of n equations given in (13), results in a system of n+1 expressions with only one unknown parameters (i.e., the tissue stiffness K). Hence, the coefficients gᵢ(d) and the force applied at the needle tip at every insertion depth d, are given by combining (13) and (14) to form the new system of equations expressed in matrix form as follows:

$$[ \begin{matrix}{g}_{1} ( d ) \\ ⋮ \\ {g}_{n} ( d ) \\ F\end{matrix} ] = \frac{K Φ}{EI Ψ + K Ω + {K}_{p} Γ + Λ} \qquad ( 15 )$$

where the matrices Φ, Ω, Γ, Φ and Λ are given by

$$Ψ = [ \begin{matrix}{ψ}_{11} & … & {ψ}_{1 n} & 0 \\ ⋮ & ⋱ & ⋮ & ⋮ \\ {ψ}_{n 1} & … & {ψ}_{nn} & 0 \\ 0 & … & 0 & 0\end{matrix} ]$$

$$Ω = [ \begin{matrix}{ω}_{11} & … & {ω}_{1 n} & 0 \\ ⋮ & ⋱ & ⋮ & ⋮ \\ {ω}_{n 1} & … & {ω}_{nn} & 0 \\ 0 & … & 0 & 0\end{matrix} ]$$

$$Γ = [ \begin{matrix}{γ}_{11} & … & {γ}_{1 n} & 0 \\ ⋮ & ⋱ & ⋮ & ⋮ \\ {γ}_{n 1} & … & {γ}_{nn} & 0 \\ 0 & … & 0 & 0\end{matrix} ]$$

$$Λ = [ \begin{matrix}0 & … & 0 & - 1 \\ ⋮ & ⋱ & ⋮ & ⋮ \\ 0 & … & 0 & - 1 \\ 1 & … & 1 & 0\end{matrix} ]$$

$$Φ = [ {φ}_{11} … {φ}_{n 1} {v}_{L} ]$$

Notice that all matrices but Φ in the previous equation are n+1 square defined. Now, needle deflection can be calculated for every insertion depth using (1).

ii. Needle—Tissue Interaction Model 2

In order to calculate the force F applied at the needle tip, the needle steering apparatus measures the forces applied to the needle's base Fᵢₙ that are necessary to insert and withdraw it from the tissue. As the needle is pushed into tissue, a force F_{c} is applied at the needle tip, that has transverse and longitudinal components Q, and F, respectively. These forces are functions of F_{c} and of the needle bevel angle β. As the surgeon pushes the needle into the tissue, the measured force at the needle base Fᵢₙ corresponds to F₁=P+f where f is the needle-tissue frictional force along the shaft given by f=(bv₁)d, where v₁ is the insertion velocity, and b is the friction coefficient per unit length of the inserted needle. When the needle is withdrawn after insertion, the measured force F₂ corresponds to friction only. If the needle is withdrawn with a velocity of v₂, the force P can be found as

$$P = {F}_{1} - {F}_{2} ( \frac{{v}_{1}}{{v}_{2}} ) \qquad ( 16 )$$

It is thereby implied that b is constant during insertion and withdrawal. The force F is finally computed as F=P(tan β)⁻¹, where β is the needle bevel angle. Knowing F, one can determine K by fitting the model such that the estimated needle deflection \hat{v}ᵢ(K) matches the measured deflection vᵢ of an inserted needle, at a point i along its shaft. More specifically, K is found to minimize

$$J ( K ) = min {\sum }_{i = 1}^{n} {( {v}_{i} - {\hat{v}}_{i} ( K ) )}^{2} , \qquad ( 17 )$$

where n is the number of measurements taken.
Once the needle-tissue model parameters are identified, the model can be used to estimate the optimal needle rotation depths.

### Needle Steering Control Algorithm

An object of the computer-implemented system and method of the present invention is to determine control commands to be applied to the device so that the needle moves along a target needle trajectory. It will be appreciated that needle steering control algorithms other than the particular model 1 and 2 described below may be suitable for use with the present invention so long as the model allows for the calculation of the amount by which the needle must be axially rotated to reach a desired target. For example, in embodiments, the needle steering control algorithm may continuously determine the amount of rotation required as the needle insertion depth varies. Alternatively, the needle steering control algorithm may determine the depth(s) at which the needle must be rotated by a discrete amount (e.g., 180 degrees) by the hand-held device in order to reach a desired target.

### i) Needle Steering Control Algorithm 1

In the Exemplary Embodiment Described Below, the Steering Algorithm Works in Three distinct phases as follows to determine the needle rotation depths at which the needle is rotated by the discrete amount.

Phase 1—Observation Phase

The ultrasound probe has moved in synchrony with the needle tip up to a certain insertion depth d enabling the model to predict both the needle deflection and the force applied at the needle tip F′ using (15). At this stage, the current needle shape, the estimated F′, and the path cut by the needle tip are known. This information is used in Phase 2 in order to predict the needle deflection as the needle is inserted further into tissue.

Phase 2—Prediction Phase

Phase 2 predicts the needle deflection for upcoming insertion depths. Unlike in Phase 1, for causality reasons the force applied at the needle tip cannot be directly observed, nor can any image feedback be obtained. Therefore, in order to calculate future needle deflections, we use (13) and set F=F′u(d), with F′ being the average estimate from Phase 1. u(d) is an auxiliary variable to reverse the orientation of the tip force when the needle base is axially rotated by 180 degrees at a depth dᵣ, and given by

$$u ( d ) = H ( d ) + 2 {\sum }_{r = 1}^{N} {( - 1 )}^{r} H ( d - {d}_{r} ) \qquad ( 18 )$$

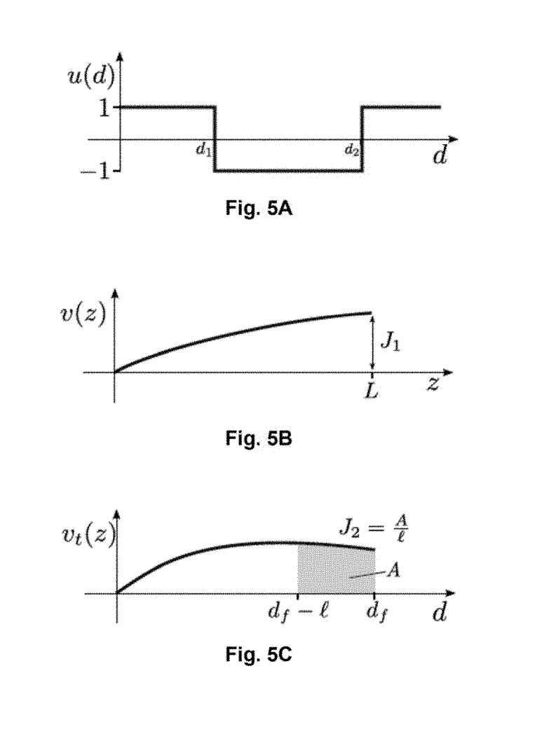

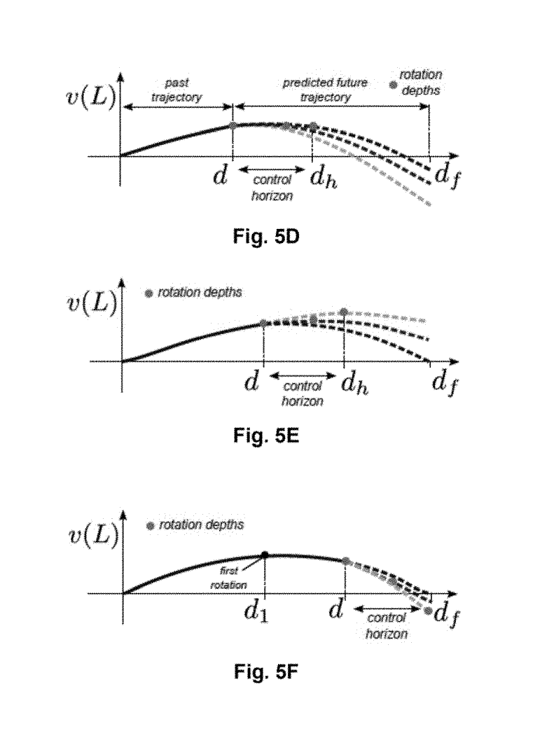

with H(d) being the Heaviside step function, and N number of admissible axial needle rotations. As shown in FIG. 5A, u(d)=1 indicates that the needle bevel tip is facing up, causing the needle to deflect downward (as shown in FIG. 4), and u(d)=−1 indicates the bevel angle is facing down, causing the needle to deflect upwards. The sign of the tip force F is then reversed every time the needle passes through a depth dᵣ, with 1≤r≤N and r∈N.

Phase 3—Control Phase

Now, the role of the steering algorithm is to find the N needle rotation depths dᵣ that minimize a cost function J representing the total needle targeting error relative to the desired target/trajectory. For formulating J, let us consider two different procedures commonly used in brachytherapy seed implantation.

Case 1:

In this experimental scenario the needle is loaded with a single radioactive seed, which must be deposited at a certain target depth in tissue, called d_{f}. Thus, the needle tip should reach the target regardless of what trajectory the needle takes. This case can also be useful for tissue biopsy. Since in brachytherapy, the needle insertion point and the target are typically on the same horizontal line, the cost function essentially amounts to minimizing the needle tip deflection at the depth of the target (see FIG. 5B). Hence, the cost function is defined as

J₁=|v(d_{f},L)|. (19)

(19)

Case 2: As in current low-dose rate (LDR) brachytherapy, several seeds spaced appropriately can be loaded in the same needle prior to insertion.

Once the needle reaches the target depth d_{f}, the surgeon holds the stylet in place and withdraws the needle such that all the seeds are deposited along the prostate length, denoted by l. Ideally and according to the dosimetry pre-planning assumptions, this chain of seeds will wind up on the horizontal line that connects the target depth to the insertion point in tissue. Thus, the cost function is defined as the mean absolute error of tip deflection inside the prostate (see FIG. 5C). Hence, in this case the cost function J₂ is defined as

$${J}_{2} = \frac{1}{𝓁} {\sum }_{d = {d}_{f} - 𝓁}^{d = {d}_{f}} \vert v ( d , L ) \vert . \qquad ( 20 )$$

The optimal depths d at which the device must rotate the needle are those that minimize the cost function for each scenario over a fixed control horizon. Inspired by Model Predictive Control (MPC) theory, the control horizon is defined as a moving window that starts at the current insertion depth and ends at a pre-defined future depths (35 mm ahead). This will correspond to the spatial interval in which the optimization solver tries to minimize the cost function. Thereby, we convert the N-variable optimization problem into a single variable optimization problem. Optimization is performed by a simulated annealing algorithm [22]. This solver provides a fast minimization of a quadratic function subject to linear and nonlinear constraints and bounds.

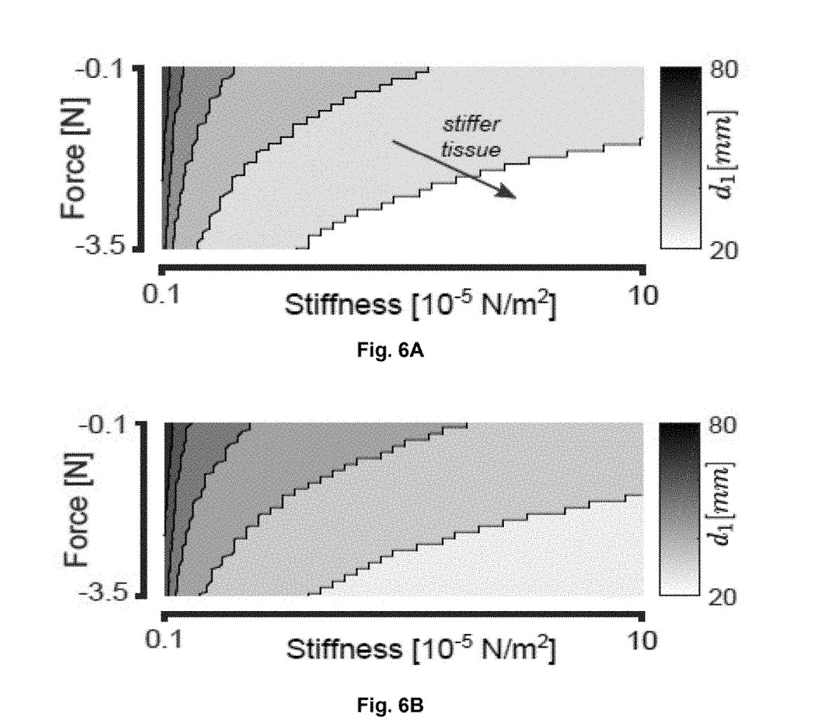

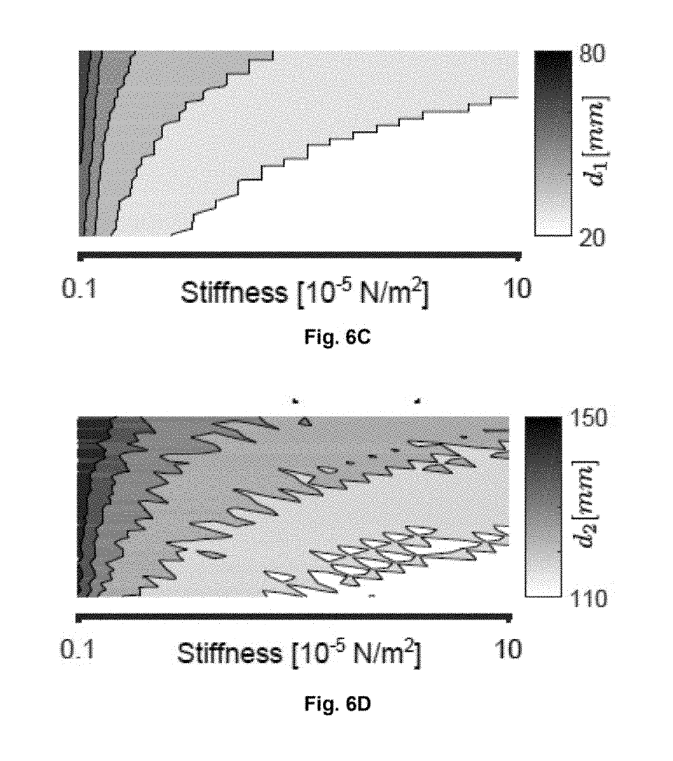

FIG. 5D shows a hypothetical example for Case 1, where the needle is first inserted to a depth d. The controller evaluates the future needle deflection up to the target depth d_{f} for different rotation depth candidates sitting within the control horizon [d; dh], where dₕ=d+35 mm. In the current control horizon, the optimal depth for rotation is determined by the optimize to be dₕ. In FIG. 5E, the needle is further advanced into tissue. Whenever the updated optimal rotation depth becomes equal to the current insertion depth, the needle must be rotated right the way. The optimal rotation depths calculated using the proposed algorithm for an 18-gauge standard brachytherapy needle (whose characteristics will be given in the next section) inserted in different tissues with stiffness per unit length K values ranging from 0.1×10⁵ to 10×10⁵ Nm⁻² and experiencing a force at the tip of 0.1≤|Fj|≤3.5 N are presented in FIGS. 6A-D. In FIG. 6A and FIG. 6B the target is a single point that is at a depth of 150 mm and 130 mm, respectively (Case 1). These results indicate that a single needle rotation is required to minimize J₁ and reach the target. For Case 2, the model predicts that two rotations are necessary to minimize the cost function J₂. The corresponding depths of the first (d₁) and second (d₂) rotations are shown in FIG. 6C and FIG. 6D for a target depth of 150 mm.

### ii) Needle Steering Control Algorithm 2

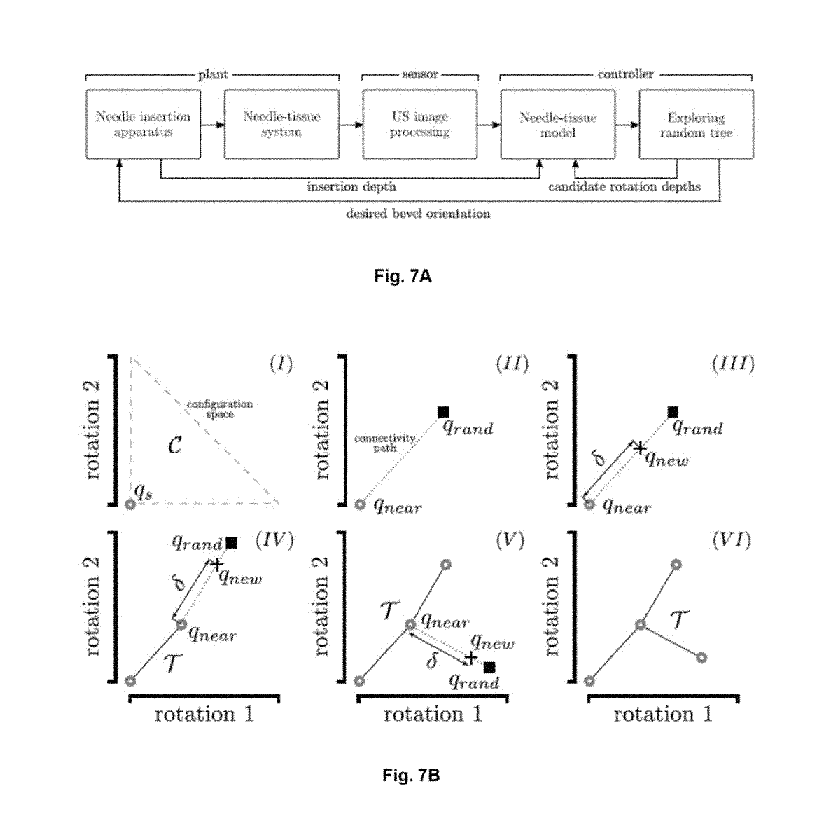

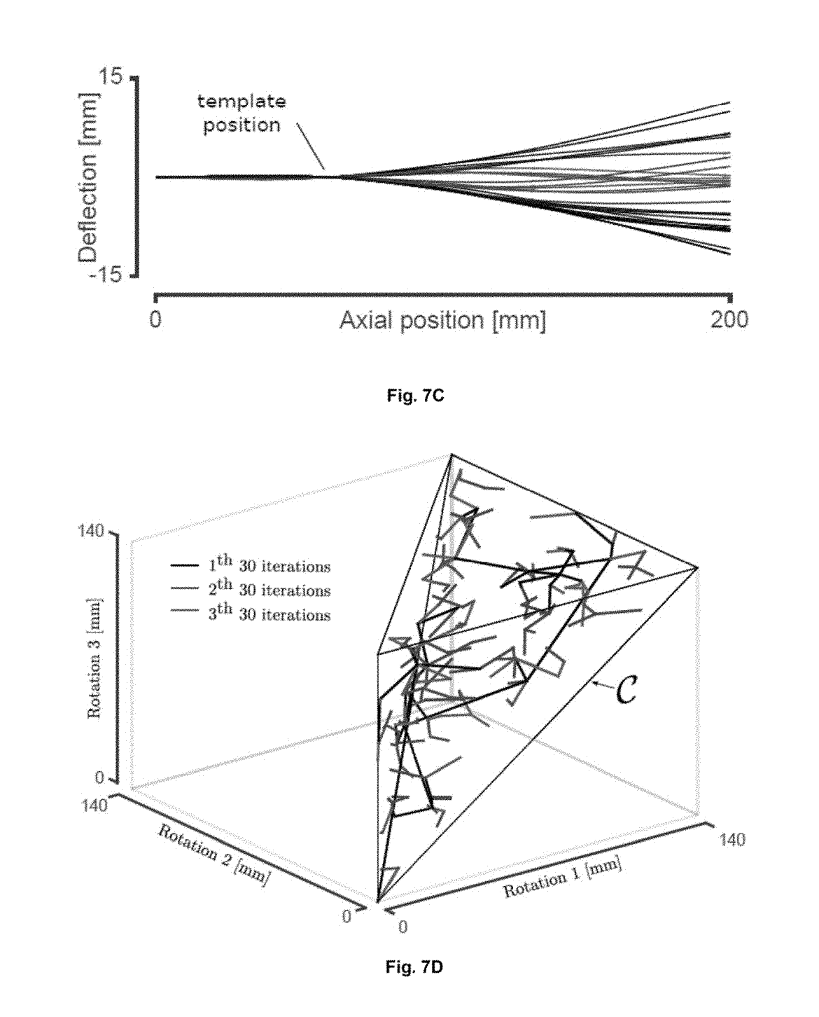

A motion planner computes a large number of needle tip trajectories (plans) using the model presented in [33] and selects the best plan. It outputs a set of depths at which the needle is axially rotated that brings the needle to the target. The planner uses the Rapidly Exploring Random Tree (RRT) algorithm [34, 35] to calculate the rotation depths. RRT is an efficient sampling algorithm to quickly search high-dimensional spaces that have algebraic constraints such as the number of allowed needle rotations, by randomly building a space-filling tree. FIG. 7A shows a block diagram of the closed-loop control algorithm based on the online motion planning.

To design the online motion planner we present the needle steering problem in the needle configuration space, called C. Assuming the needle moves in a 2D insertion plane, the needle workspace is a Euclidean space W=R². The configuration space (C) is the space of all possible control actions (i.e., depth(s) of needle rotation(s)), whose values identify the configuration of the needle tip in the workspace. Considering symmetry of rotation depths (e.g., rotations at depths of 40 and 80 mm are equal to rotations at 80 and 40 mm) the configuration space is an n-dimensional simplex, where n is the number of axial rotations. For instance, if the maximum allowable number of rotations is 3, the configuration space forms a tetrahedron.

The proposed motion planner uses an approximate decomposition of C. Assuming that the distance between two consecutive rotations is at least 5 mm, C can be decomposed into several smaller simplices shown in FIG. 7C. This is a valid assumption since two close 180° axial rotations are equal to one 360° rotation of the needle tip and this action has no effect on needle deflection. The disjoint cells in C form a connectivity graph. The nodes of this graph are vertices of the cells corresponding to a certain configuration (i.e., rotation depths). Assuming that the initial guess for a configuration in C is qₛ and the goal configuration that steers the needle toward the target is q₂, planning a motion for the needle involves searching the connectivity graph for a path from cell containing qₛ to the cell containing qₛ. For this purpose we use the RRT algorithm. In the following a pseudocode description of the motion planner algorithm is given.

Algorithm 1: q_{goal} ← RRT_Algorithm
(X₀,N,Tₘₐₓ)

```
C ← Initialize_space (X₀, N)
T ← Initialize_tree (X₀,N)
while X = ø ∧ Γ < Γₘₐₓ do
  |  q_{rand} ← Rand_Conf (C)
  |  qₙₑₐᵣ ← Near_Vertex (q_{rand}, C)
  |  q_{new} ← New_Conf (q_{rand}, qₙₑₐᵣ)
  |  P_{new} ← Needle-tissue-model[27]
  |  (q_{new})
  |  T ← Add_Vertex(q_{new})
  |  T ← Add_Edge(q_{new}, qₙₑₐᵣ)
  |  if p_{new} ∈ [symbol]   then
  |   |  q_{goal} ← Extract_Conf(q_{new})
  |  end
end
```

The inputs of the RRT are the current depth X₀, the number of allowed rotations N, and the computation time available for planning Tₘₐₓ. A hypothetical example of tree generation for N=2 is shown in FIG. 7B. First, the configuration space C is formed based on the number of allowed rotations N and the current needle insertion depth X₀=0. The tree is initialized with a first vertex qₛ located at (0, 0) (see (I) in FIG. 7B). The algorithm then generates a random candidate q_{rand}, from the N-dimensional configuration space C (See Rand_Conf in Algorithm 1, and (II) in FIG. 7B). Next, Near_Vertex runs through all the vertices (candidate rotation depths) in C to find the closest vertex to q_{rand}. New_Conf produces a new candidate configuration q_{new} on the segment joining qₙₑₐᵣ to q_{rand} at a predefined arbitrary distance δ from qₙₑₐᵣ (see (III) in FIG. 7B).

The random tree T is expanded by incorporating q_{new} and the segment joining it to qₙₑₐᵣ, as shown in (V1) in FIG. 7B. Next, needle tip path and targeting accuracy (p_{new}) are obtained by inputting the selected rotation depths in the needle-tissue interaction model [33]. The predicted needle shape for various candidate set of rotation depths is shown in FIG. 7C. When the needle path for the newly added configuration is found to lie in the target region (G), or when the computation times exceeds Tₘₐₓ the RRT planner terminates. The target region is a closed circle with 1 mm diameter, centred on the desired target location in W. The former condition implies that when the estimated needle tip deflection at the maximum depth is less than 0.5 mm, the algorithm stops. If the stopping condition is not met, the algorithm continues to expand the tree with new vertices as depicted in (V) and (V1) in FIG. 7B.

Once the algorithm stops, the output q_{goal} contains the best set of rotation depths that will bring the needle towards G. The RRT expansion procedure results in a very efficient exploration of C and the procedure for generating new candidates in RRT is intrinsically biased toward regions of C that have not been visited.

In prostate brachytherapy, the needle insertion point and the target are typically on the same horizontal line. The target is assumed to lie at a depth of 140 mm. In order to limit tissue trauma, the total number of needle axial rotations is set to three. Results of the simulation of the motion planner in configuration space C and the corresponding needle deflection predictions in needle workspace W for an insertion depth of 140 mm starting at 0 mm are shown in FIGS. 7C and 7D, respectively.

The RRT has been used for needle steering in [34]. Unlike [34], our search space is directly constrained by the possible control inputs and by the number and depths of rotations. Therefore, there is no need to solve for the inverse kinematics of the model, which enables the optimization problem to be solved faster and makes the solution method suitable for online applications.

### System

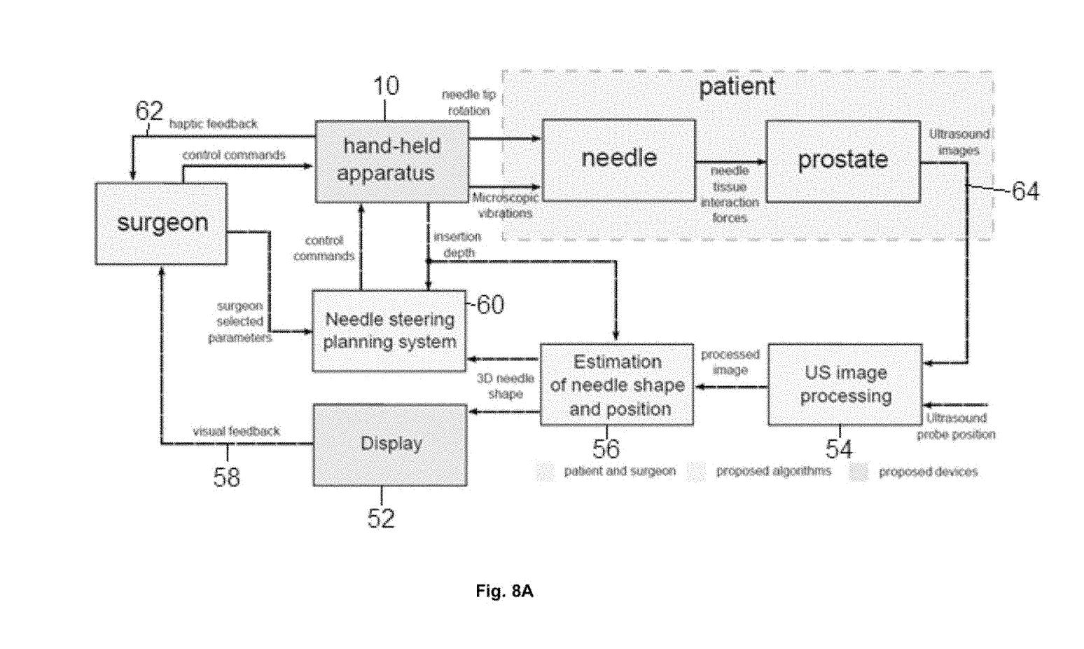

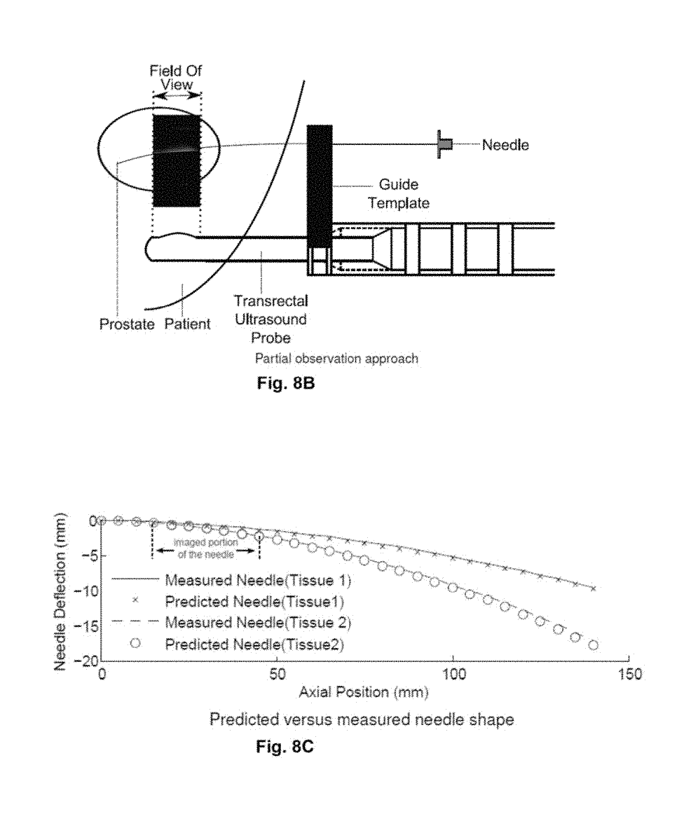

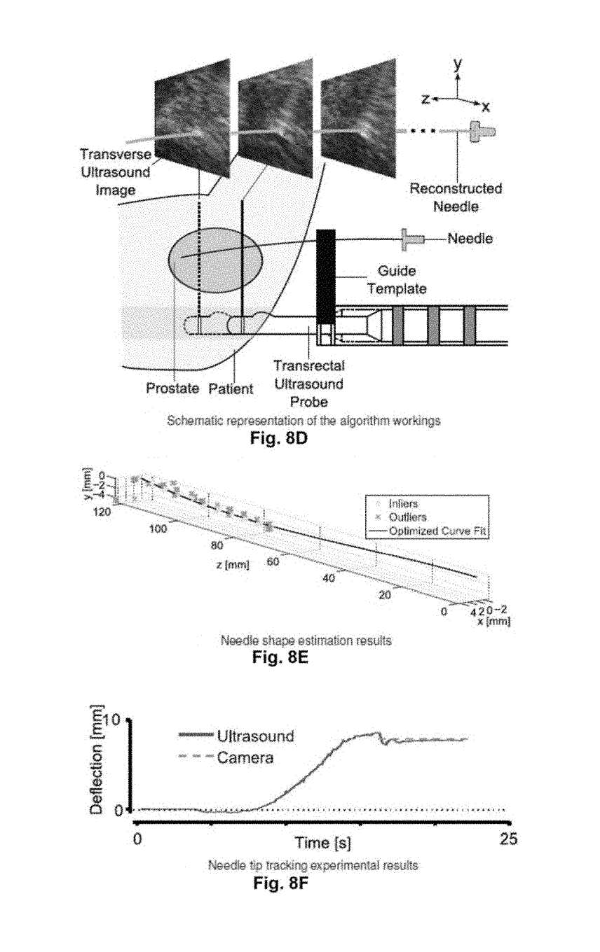

In the exemplary embodiment shown schematically in FIG. 8A, the system of the present invention comprises the hand-held device 10 of the present invention, a display device 52, and a computer operatively connected to the hand-held device 10 and the display device 52, in relation to a surgeon, a patient, and a transrectal ultrasound probe. It will be understood that the operative connections between the computer, the hand-held device 10, and the display device 52 may comprise wired connections, wireless connections or a combination of wired and wired connections.

### Display Device

A purpose of the display device 52 is to generate visual representations of the information relevant to needle insertion, such as needle shape, needle position or needle insertion parameters such as a needle position, a needle orientation, a needle axial rotation angle, a needle velocity, and a needle acceleration, wherein the calculating is based on an electronic signal from the sensor unit 20 and the ultrasound images. The information may be displayed numerically and/or by graphical representations. In exemplary embodiments, the display device 52 may comprise one or a combination of a video display screen.

### Computer

A purpose of the computer is to control the device and the display device in accordance with methods of the present invention, for assisting the surgeon to precisely and efficiently place the needle. In general, the computer comprises a computer processor and a computer memory. In an exemplary embodiment, the computer processor may comprise a microprocessor (i.e., a computer processor on an integrated circuit device). The computer processor executes the instructions stored on the computer memory to implement methods of the present invention. The computer memory is a computer device that comprises a non-transitory computer readable medium that stores instructions that are executable by computer processor to implement methods of the present invention. In exemplary embodiments, the computer memory may comprise volatile memory (i.e., memory that requires power to maintain the stored data) as well as non-volatile memory (i.e., memory that can be retrieved after power to the computer memory has been cycled on and off). In exemplary embodiments, the computer memory may comprise solid-state flash memory, magnetic media, and optical media. It will be appreciated that the computer may be implemented by one or more general purpose computers, a special purpose computers, or a combination of general purpose computers and special purpose computers with appropriate software or firmware stored on a variety of non-transitory computer readable media, as known to persons skilled in the art. The computer may be partly or wholly physically integrated with or physically discrete from the hand-held device.

The computer may be characterized as having functional modules including a ultrasound image processing module (USIPM) 54, a needle shape and position estimation module (NSPEM) 56, a display module (DM) 58, a needle steering planning module (NSPM) 60, and a haptic feedback module (HFM) 62. It will be appreciated that these functional modules are not physically discrete modules, and may functionally overlap with each other in operation.

A purpose of the ultrasound image processing module (USIPM) 54 is to process an ultrasound image 64 to determine a location of a portion of the needle 12 within the ultrasound image 64. A purpose of the needle shape and position estimation module (NSPEM) 56 is to calculate a shape and position of the of the needle 12 based on locations of known portions of the needle 12 as determined by the USIPM 54, and/or needle insertion parameters derived from electronic signals generated by the sensor unit 20 of the hand-held device 10. When using a transrectal ultrasound probe, it may be desirable to use a thin, firm sleeve that minimizes prostate deformation as the probe is moved inside the rectum. Alternatively, it may be desirable to use an ultrasound system such as TargetScan (Envisioneering Medical, Pittsburgh, USA) or the anorectal 3D 2052 ultrasound probe (BK Ultrasound Machines, Peabody, Mass., United States), in which the probe is stationary inside the rectum, but the transverse imaging plane can be changed.

In an exemplary embodiment as shown in FIG. 8B, the needle 12 is imaged in the sagittal ultrasound plane, where only a small portion (roughly 40 mm) of the needle 12 is visible as shown schematically in FIG. 8B. This is input into the needle-tissue interaction model and the observation phase of the needle steering control algorithm. This technique allows the ultrasound probe to remain stationary during brachytherapy, which will eliminate any ultrasound probe induced tissue motion. The points immediately above “imaged portion of the needle” in FIG. 8C, represent the portion of the needle viewed in the ultrasound image. The error in predicting the entire needle shape is less than 0.5 mm. This technique uses only a frame grabber device connected to an ultrasound imaging system and does not require any modification to the clinical ultrasound machine. The ultrasound images may be captured or “grabbed” at a rate of approximately 30 to 60 frames a second.

In an alternative exemplary embodiment as shown in FIG. 8D, the needle shape and position is estimated using a series of transverse images of the needle 12 obtained at different depths. An image processing routine that enhances the visibility of the needle 12 and locates candidate needle points within each of the ultrasound images. Then, using a random sample and consensus algorithm, false needle point candidates are removed and the needle shape is fit to the remaining inliers as shown in FIG. 8E. For the case where the ultrasound probe moves at the same velocity as the needle during insertion, the success of the real-time needle deflection estimation algorithm is shown in FIG. 8F. Coordination of the velocity of the ultrasound probe and the needle 12 may be accomplished by physical connection between the ultrasound probe and the hand-held device 10, or controlling movement of the ultrasound probe using information derived from the sensor unit 20 of the hand-held device 10.

A purpose of the display module (DM) 58 is to cause the display device 52 to show the needle shape and position, and/or needle insertion parameters determined by the NSPEM 56.

A purpose of the needle steering planning module (NSPM) 60 is to predict the needle trajectory, calculate a correction to one or more needle insertion parameters for a target needle trajectory including at least a correction to the needle axial rotation angle paired with a needle rotation depth. In an exemplary embodiment, these calculations may be made in accordance with the prediction phase and control phase of the needle steering control algorithm. Having determined the correction to the needle insertion parameter, in an exemplary embodiment, the NSPM 60 controls the rotary actuator 34 of the hand-held device to axially rotate the needle 12 by the correction to the needle axial rotation angle at the paired needle rotation depth.

A purpose of the haptic feedback module (HFM) 62 is to activate the haptic feedback unit 22 of the device 10 to vibrate the handle 16 of the device 10 in a vibration pattern to provide a tactile alert to the surgeon when the NSPM 60 determines that corrections to the needle insertion parameters are required or not required. In an exemplary embodiment, the vibration pattern is determined by a rules database depending on one or a combination of the calculated needle shape, the calculated needle position, and the calculated correction to the needle insertion parameter. For example, the rules database may cause the HFM 62 to activate the haptic feedback unit 22 to generate vibration patterns that are recognizably distinct to the surgeon (e.g., vibration patterns characterized by different durations, amplitudes, and/or number of vibrations) that correspond to the need or lack of need for different corrective needle steering maneuvers (e.g., accelerate/decelerate needle insertion; rotate needle clockwise/counter-clockwise; push needle up/down/left/right; pause needle motion; maintain needle motion), or to the arrival of the needle at the target position.

### Applications

As described above, the present invention provides real-time assistance to a surgeon to precisely, efficiently, and intuitively position a needle percutaneously inserted in a patient to achieve a target needle trajectory, while allowing the surgeon to maintain at least partial manual control over needle insertion. In an exemplary embodiment, the invention may be used during prostate brachytherapy. However, it will be appreciated that the invention may be used for brachytherapy treatments for organs including, but not limited to, prostate, breast, cervix, and skin, and tumors in other body sites.

### Example 1

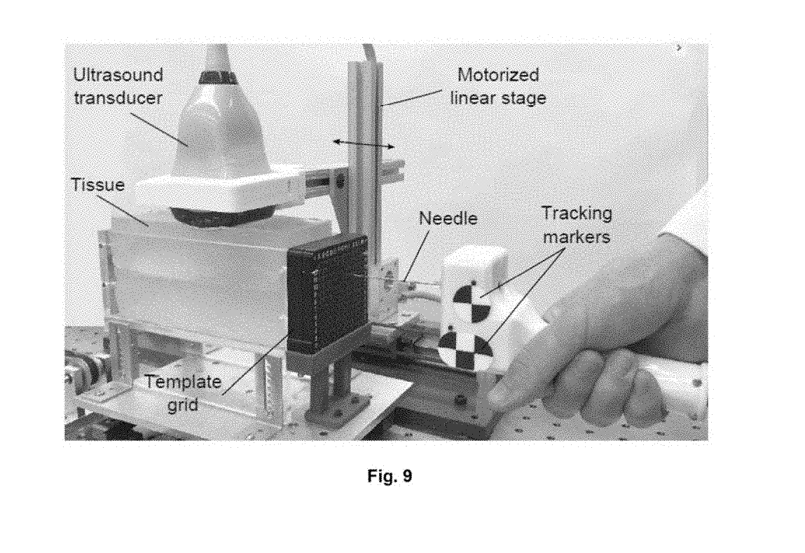

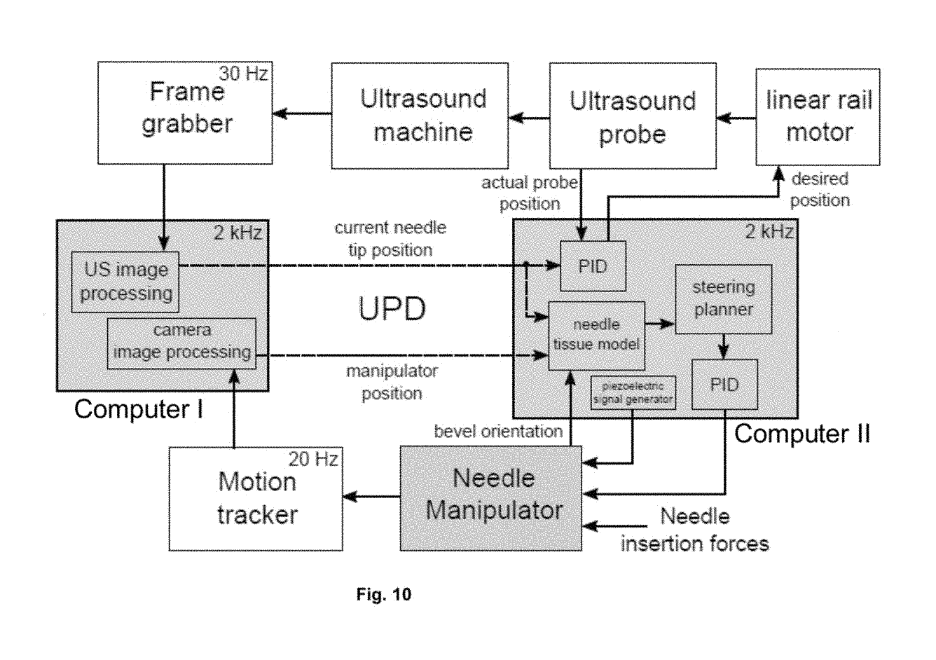

The experimental setup used to test the prototype system is shown in FIG. 9 in phantom and ex-vivo biological tissue. A schematic depiction of the prototype system is shown in FIG. 10. In order to track the position of the handle of the device in real time, the 3D position of tracking markers added to its left is measured at 20 Hz by a dual camera optical motion tracker (BB2-BWHx60 from Claron Tech, Toronto, Canada). The needle is inserted by the handheld assistant into a piece of tissue held in a transparent container through a standard brachytherapy template grid (model D0240018BK, C.R. Bard, Covington, USA). The grid template is assumed to have a stiffness of Kp=10⁹ Nm⁻¹. From the measured position of the tracking markers and knowing the length of the needle, the needle insertion depth is deduced. The needle used throughout the experiments is a 200 mm long 18-gauge standard brachytherapy needle (Eckert & Ziegler Inc., Oxford, USA) with a Young's modulus of 200 GPa and a moment of inertia of 7.75×10⁻¹⁴ m⁴.

As the needle is inserted in the tissue, a 4DL14-5/38 linear ultrasound probe connected to a Sonix Touch ultrasound machine (Ultrasonix, Richmond, Canada) slides above the tissue to acquire at 30 Hz transverse 2D ultrasound images of the needle. Transverse images show a cross section of the needle ensuring that the problem of probe alignment found in longitudinal (sagittal) imaging will not be present [23]. A linear stage motorized by a DC motor moves the ultrasound probe, while its absolute position is measured by a linear potentiometer (LP-250FJ from Midori Precisions, Tokyo, Japan) in real time (not visible in FIG. 9). The ultrasound imaging plane is initially placed close to the needle tip. As the needle is pushed into the tissue, the motorized linear stage controlled by a discrete PID (proportional-integral-derivative) controller moves in synchrony with the needle tip such that the same point of the needle shaft is always visible in the image. Each transverse ultrasound image is then processed in order to obtain the current needle tip deflection using the algorithm presented in [23]. For safety reasons, the motorized linear stage that translates the ultrasound probe is only activated when the needle is inserted through the grid template. This is done by establishing a virtual workspace defined as a 3D rectangular volume located in front of the grid template. For the hand-held device to be in that workspace, the needle must be inserted in the grid template.

Two computers running Matlab™ in xPC real-time mode are used in the experimental setup as depicted in FIG. 10. Computer I receives images generated by the ultrasound machine which are captured by a frame grabber, and images of the tracking markers obtained by the motion tracker. After processing the images, the measured needle tip deflection in the current ultrasound image and the 3D coordinates of the hand held device are sent via UDP (user datagram protocol) to Computer II. The current needle tip position can then be used in Computer II by the needle-tissue model and the steering algorithm. A PID compensator controls the desired orientation of the needle bevel angle calculated by the steering algorithm. The position of the hand-held device is sent to a second digital PID compensator that adjusts the horizontal position of the ultrasound imaging plane. Both control loops run at 2000 Hz and the communication delay between them is 4 ms.

We performed needle insertion in three different tissues. Tissue 1 and Tissue 2 are made of industrial gelatin derived from acid-cured tissue (gel strength 300 from Sigma-Aldrich Corporation, Saint Louis, USA). The mass ratio of gelatin to water in Tissue 1 and Tissue 2 is 0.15:1 and 0.2:1, respectively, making Tissue 2 stiffer than Tissue 1. Tissue 3 is prepared by embedding a 130 mm long piece of beef tenderloin in the same gelatin used in Tissue 2. This tissue presents several layers of fat and muscle, making it highly non-homogeneous. The gelatin is meant to create a flat surface to ensure good acoustic contact between the ultrasound probe and the biological tissue and to generate a second thin tissue layer. In the experiments in Tissue 3, the needle first goes through the biological tissue. When the insertion depth is higher than 130 mm, the needle reaches the gelatin layer. For each of the three tissue samples and two steering cases, we carried out needle insertions to attain two different target depths i.e., 130 mm and 150 mm. This amounts to a total of 12 different experimental scenarios. For each scenario, 6 needle insertions were performed, which yields a total of 72 needle insertions.

The following discussion is divided in three parts. First, we will see the effects of longitudinal micro vibrations cause by the piezo-actuator on needle-tissue friction. Next, image-based identification of needle tissue interaction model parameters are described. The obtained results are used to steer the needle towards pre-determined targets.

A. Effects of Longitudinal Needle Vibration

In order to observe the effects of needle longitudinal vibration on the needle-tissue frictional forces, the piezoelectric actuator unit connected to an 18-gauge brachytherapy needle is attached to the needle insertion robot presented in [24]. The robot is controlled to insert the needle at a constant insertion velocity of 5 mm s⁻⁴ through a 40 mm thick piece of tissue made of plastisol gel (M-F Manufacturing Co., Fort Worth, USA) with a Young's modulus of 25 kPa. Once the needle tip is placed close the tissue surface, the robot is controlled to move the needle towards the tissue a distance of 70 mm, while the axial insertion force is recorded by a force sensor.

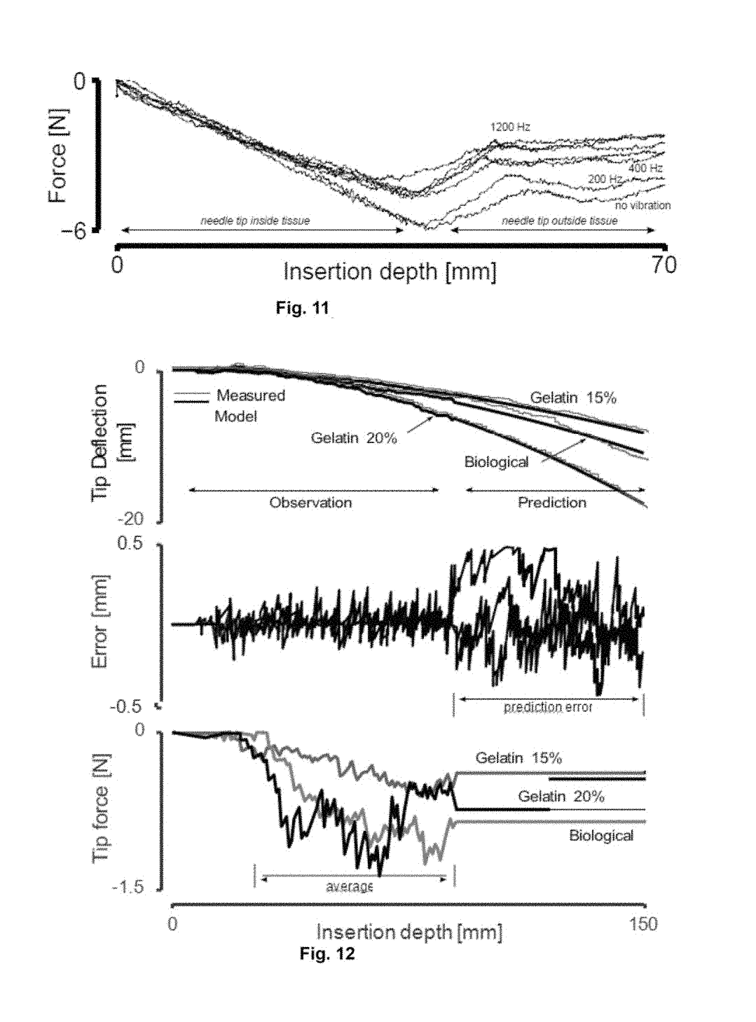

As the needle tip passes through the tissue, the measured force corresponds to the axial needle-tissue cutting force plus the frictional force generated along the shaft. Inertial effects are neglected since the needle is driven at a constant velocity. When the needle tip exits the tissue, the measured force corresponds to friction only. For each insertion, the piezoelectric actuator receives a 5 V in amplitude sinusoidal voltage with a different frequency ranging from 0 Hz (no vibration) to 1200 Hz in 200 Hz increments. The measured insertion force for each frequency is presented in FIG. 11. The results show that the needle insertion forces can be reduced up to 48%. No considerable variation in the insertion force is observed for frequencies beyond 1200 Hz.

B. Model Parameterization from Ultrasound Images

The first step towards needle steering is to find the model parameters, i.e., the tip force F and the needle tissue stiffness. To this end, three insertions are performed in each tissue without axial rotation. From the acquired data, and using the needle tissue interaction model, these parameters can be calculated. Next, the optimal depths of rotation can be calculated for each experimental scenario as described in the needle steering control algorithm above. The model parameters are found following each of the 7 steps detailed below.
1) Insert the needle in tissue and record the needle deflection using ultrasound images (see the first plot in FIG. 12);
2) In the needle-tissue model, initialize or, whenever appropriate, update the current needle-tissue stiffness K;
3) Run the observation phase up to 60% of the maximum insertion depth;
4) Calculate the average of the observed force F during the observation phase (see the third panel in FIG. 12. Due to imaging noise, the first 20 mm are not considered);
5) Using the average force F from Step 4 and the current stiffness K from Step 2, run the prediction phase from the end of the observation phase to the maximum insertion depth;
6) Evaluate the mean squared error between the model predicted and measured needle tip deflection (see the second panel in FIG. 12);
7) Repeat the process from Step 2 until the prediction error in Step 6 reaches a minimum.

FIG. 12 shows the estimated tip deflection for each tissue sample and after model parameterization. The prediction error for both phantom tissues is less than 0.2 mm, and increases to 0.5 mm for the biological tissue. The obtained model parameters are summarized in the first two lines of Table I.

TABLE I
IDENTIFIED MODEL PARAMETERS FOR
EACH EXPERIMENTAL SCENARIO

| | Gelatin 15% | Gelatin 20% | Biological |
|---|---|---|---|
| Stiffness K [N m⁻²] | 0.5 × 10⁵ | 1.2 × 10⁵ | 1.6 × 10⁵ |
| Estimated force F [N] | −0.33 | −0.85 | −0.73 |
| Measured force F [N] | −0.39 | −0.96 | −0.57 |

For comparison, the third line shows the tip force F measured for each tissue by means of a force sensor connected to the needle's base and the procedure described in [24]. Note that different combinations of stiffness K and tip force F can lead to the same tip deflection at a given depth. The disparity between them can be seen in the path followed by the needle tip (i.e., vₜ(d,z)). Hence, a tissue with high K−F does not necessary have a high Young's modulus, but rather will induce the needle to deflect forming a high radius of curvature (tending to a straight line). This is the case for the biological tissue as compared to the gelatin phantom tissues.

C. Needle Steering

Updating the model parameters as the needle is inserted requires the ultrasound probe to move in synchrony with the needle tip during the procedure [25], [26]. However, automated ultrasound probe motion is rarely available in operating rooms. Furthermore, probe motion during brachytherapy can result in additional deformation of the prostate gland [27]. This has been shown to result in anatomic variations of the preoperatively planned needle targets [28], [29]. Hence, it is desirable to limit the motion of the ultrasound probe. For these reasons, the experiments reported here assume that the identified model parameters are constant during insertion and the steering algorithm does not employ ultrasound images during insertion. Six needle insertions are performed for each experimental scenario using the hand-held device. Each insertion is done at a new location in tissue to avoid the influence of previous insertions on the current one. Table II shows the calculated optimal depth(s) (i.e., d1 and d2, if applicable) where the needle rotates by 180 degrees during insertion.

TABLE II
MEASURED AVERAGE NEEDLE TIP DEFLECTION (J₁) AT A DEPTH d [symbol] AND AVERAGE NEEDLE
TIP DEFLECTION (J₂) BETWEEN d [symbol] AND d [symbol] - 50 FOR EACH EXPERIMENTAL
CONDITION. ALL UNITS ARE IN MILLIMETERS.

| | Target | Tissue | Rotation | Rotation | Cost | J₁ stand. | Cost | J₂ stand. | Prediction |
|---|---|---|---|---|---|---|---|---|---|
| Case | depth d [symbol] | sample | depth d₁ | depth d₂ | function J₁ | deviation | function J₂ | deviation | error |
| 1 | 150 | Gelatin 15% | 59 | n.a. | 0.33 | 0.38 | 1.06 | 0.36 | 0.33 |
| | | Gelatin 20% | 56 | n.a. | 0.27 | 0.28 | 1.19 | 0.32 | 0.27 |
| | | Biological | 48 | n.a. | 0.32 | 0.32 | 0.96 | 0.48 | 0.32 |
| | 130 | Gelatin 15% | 53 | n.a. | 0.35 | 0.39 | 1.02 | 0.63 | 0.35 |
| | | Gelatin 20% | 50 | n.a. | 0.54 | 0.21 | 1.53 | 0.21 | 0.54 |
| | | Biological | 42 | n.a. | 0.77 | 0.51 | 0.92 | 0.37 | 0.77 |
| 2 | 150 | Gelatin 15% | 50 | 138 | 0.42 | 0.28 | 0.61 | 0.53 | 0.31 |
| | | Gelatin 20% | 41 | 123 | 0.46 | 0.34 | 0.27 | 0.54 | 0.13 |
| | | Biological | 38 | 121 | 0.76 | 0.31 | 0.45 | 0.25 | 0.37 |
| | 130 | Gelatin 15% | 45 | 122 | 0.55 | 0.14 | 0.43 | 0.50 | 0.18 |
| | | Gelatin 20% | 34 | 110 | 0.21 | 0.15 | 0.11 | 0.14 | 0.29 |
| | | Biological | 39 | 107 | 0.42 | 0.25 | 0.32 | 0.38 | 0.16 |

| | | | |
|---|---|---|---|
| Average of J₁ in Case 1, and J₂ in Case 2 | 0.43 | 0.36 | |
| Average over 72 insertions | 0.44 | 0.72 | 0.33 |

[symbol]

indicates data missing or illegible when filed

As predicted in the simulations reported in the needle steering control algorithm, the higher K, the sooner the needle is rotated. The corresponding measured values after insertion of the cost functions J1 (tip deflection at the target depth df) and J2 (average tip deflection between df−50 mm and df) are summarized in the sixth and eighth columns of Table II, respectively. Note that the objective in Case 1 is to minimize the cost function J1; J2 is only presented as an indication of the average tip deflection when the needle approaches the target depth. Likewise, in Case 2, the controller only minimizes J2. The error between the estimated and measured cost-functions is shown in the last column. For Case 1, this equals J1. For Case 2 the predicted cost function J2 is never zero due to the non-holonomic constrains of needle steering. Hence, the reported error is the difference between the model predicted and measured J2. For Case 1, the average needle tip deflection at the target depth is 0.43±0.19 mm. The highest average tip deflection is 0.77 mm, observed for biological tissue, and the lowest is 0.27 mm obtained in the gelatin tissue. With regards to Case 2, the average deflection over the 50 mm preceding the maximum depth is 0.36±0.17 mm, and the average prediction error when compared to the steering algorithm predictions is 0.24 mm. The overall average error between model predictions and the measured cost functions over 72 needle insertions is 0.33±17 mm.

D. Discussion

We have evaluated the ability of the hand-held needle steering system to minimize needle deflection in two different case studies. The first case intends to minimize the needle tip deflection at the maximum depth (quantified by J₁). The second case minimizes the needle tip deflection over the 50 mm that precede the maximum insertion depth (quantified by J2). In Case 1, J₁ does not exceed 0.7 mm while J₂ can be as high as 1.19 mm. In Case 2, J₂ is reduced to no more than 0.61 mm without affecting J₁. Hence, it can be concluded that Case 2 also contains Case 1 as a subset, at the cost of only one additional needle rotation.

Deviations between model prediction and measured results are less than 0.77 mm, with an average of 0.33 mm. This can be partially attributed to imaging uncertainties observed for model parameterization and partially to ground truth. Firstly, the ultrasound probe is imaging the needle on average 3 mm behind the needle tip, which in the worst case scenario can induce a deflection measurement error of 0.2 mm. Secondly, the noise present in ultrasound images may impair ability of the model to capture a small amount of inherent variability in the results and thereby lead to non-negligible variations in the estimated force F. The latter can be solved by improving the needle tracking algorithm or by replacing it with a more accurate measurement modality. Another source of uncertainty arises from the operator's susceptibility to involuntarily turn the wrist (rotate) as he/she uses the hand-held device. This small rotation of the needle's base can lead to a small change in the orientation of the needle bevel tip, which is not compensated for in the controller.

In spite of these uncertainties, and with a limited number of model parameters, the proposed steering system is able to steer a brachytherapy needle towards a desired target/trajectory with satisfactory accuracy. For comparison with other needle-tissue models, the nonholonomic model [15] reports an error between the model prediction and measurements of 1.3 mm. In [30] the average targeting error during steering is 0.46 mm for different kinematics and mechanics-based models. In [31], a sliding-mode based closed-loop needle steering algorithm has an accuracy of 0.43 mm. Table III shows a comparison of our proposed hand-held device with other reported models and steering algorithms.

TABLE III
COMPARISON WITH OTHER DOCUMENTED MODELS (1-2) AND
STEERING SYSTEMS (3-7)

| | | Guidance/ | Tissue | Number of | Targeting | Hand-held |
|---|---|---|---|---|---|---|
| | | parametrization | model | rotations | error | insertion |
| Abayazid et al. | [30] | US images, ARFI^{a} | soft | 2-19 | 0.46 | X |
| Webester et al. | [15] | Camera images | stiff | 1-2 | 1.30 | X |
| Rucker et al. | [31] | Magnetic tracking | stiff | 1 | 0.43 | X |
| Fichtinger et al. | [9] | CT images | soft | 2 | 1.00 | X |
| Schneider et al. | [10] | US images | rigid | 1 | 2.50 | X |
| Smith et al. | [7] | 3D US images | rigid | n.a. | 0.27 | X |
| Okazawa et al. | [13] | US images | stiff | n.a. | <1.0 | C |
| Prototype system | | US images | soft | 1-2 | 0.44 | C |

The prototype system shows fairly good accuracy when compared with other models and fully automated needle insertion schemes. Ways to improve needle targeting accuracy could be found in tracking the needle tip as it is inserted in order to update the model parameters on the fly in a closed-loop control scheme. For brachytherapy applications, this is only viable as long as the moving parts of the ultrasound probe are not in contact with the surrounding tissue. This could be implemented with a thin, firm sleeve in which a transrectal ultrasound probe translates, such that when the transducer moves, it does not deform the prostate gland and/or adjacent anatomical structures. Another option involves using an ultrasound system such as the TargetScan (Envisioneering Medical, Pittsburgh, USA) in which the probe is stationary, but the transverse imaging plane can be translated.

### Example 2

Three different tissues were used in these experiments. The first tissue was made by encasing a 130 mm long piece of porcine tissue into a mixture of 20% gelatin derived from acid-cured tissue (gel strength 300 from Sigma-Aldrich Corporation, USA) per litre of water. The gelatin was meant to create a 20 mm layer of tissue through which the needle was inserted before reaching the porcine tissue, and also to create a flat surface in order to ensure good acoustic contact between the ultrasound probe and the tissue. In the second tissue, the porcine layer was replaced with bovine tissue. Hence, the first two tissues were composed of two different layers. The third tissue was made of high friction plastisol gel (M-F Manufacturing Co., USA) mixed with 20% plastic softener. For each tissue, 15 needle insertions at different locations in the grid template followed by deposition of a single seed were performed. The seeds were deposited at a depth of 140 mm. For each tissue, a set of 15 insertions was performed using an open loop controller (image feedback is not used), and another set of 15 implants is performed using a closed-loop needle insertion controller. This amounts to a total of 6 different experimental scenarios and 90 seed implants in total.

Each seed implantation procedure was composed of three phases:

1. Phase 1—Pre-scan: The needle has not been inserted in the tissue. The ultrasound moves with a constant velocity of 8 mm·s⁻¹ up to a depth of 150 mm and returns to the initial position. Thereby, all previously implanted seeds and tracks in tissue left by other insertions can be identified.

2. Phase 2—Needle insertion: The ultrasound imaging plane is placed close to the needle tip. During insertion, the ultrasound probe moves in synchrony such that the needle tip is always visible in the image. Once the needle reaches the desired depth of 140 mm, the seed is manually deposited and the needle is withdrawn.

3. Phase 3—Post-scan: After the needle is withdrawn the tissue is scanned in order to identify the position of the seed deposited in Phase 2.

The needle steering controller may be employed in two different ways. In open-loop mode, the controller determines 3 optimal rotation depths prior to needle insertion. In closed-loop mode, the RRT controller updates the rotation online based on the measured needle tip position. The maximum computation time allowed for planning is 1 second, which was found to provide good convergence. The needle bevel angle is initially oriented such that the needle deflects in a plane that is parallel to the table shown in FIG. 9. Deflection along the vertical plane is not controlled.

Needle tip tracking is done online as the needle is inserted into the tissue. Each transverse ultrasound image is processed in real-time using the algorithm presented in [36]. Seed localization is done using the information from both the Phase 3 scan, containing the implanted seed, and the Phase 1 scan, which is used to reduce background noise in the Phase 3 transverse images. Final implanted seed positions are obtained offline after Phase 3 scan is completed. Note that when open-loop needle steering is used, the images are not used as feedback in the controller but the needle tip is still tracked.

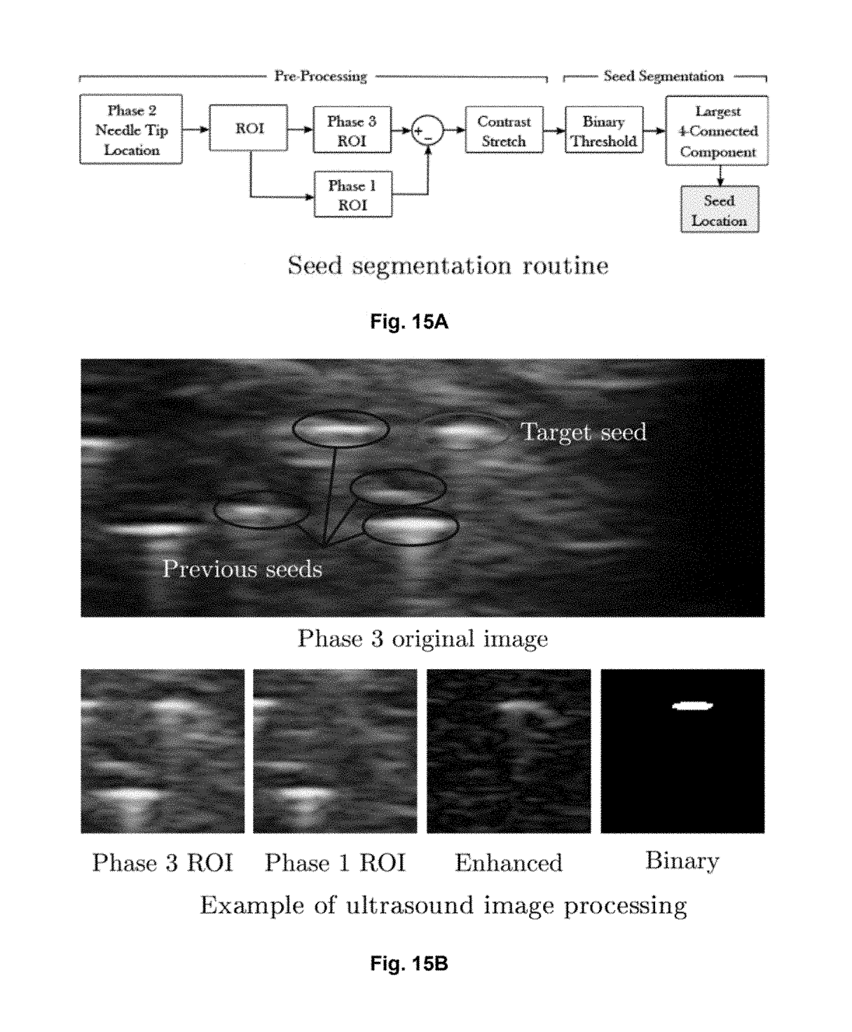

From the final needle tip position in Phase 2, the seed deposition depth is obtained and the traverse ultrasound image that contains the seed can be selected from the Phase 3 scan, which we will denote as I_{P3}. The original image obtained in Phase 3 is shown in FIG. 15B. Even with the deposition depth of the seed known, seed localization in transverse images is complicated by several factors, the most important of which is that previous seeds are present alongside the target seed, as well as the seed not being very distinct from the background image noise. An additional complication is that the implanted seed moves away from the final needle tip location, found in Phase 2, as the needle is withdrawn.

The seed tracking algorithm consists of 2 stages, i.e., a pre-processing stage and the background noise removal (FIG. 15A). The first step in the pre-processing stage is to define a region of interest (ROI) around the final needle tip location, found in Phase 2, in I_{P3} that is large enough to capture the seed with moderate motion. Empirically, an ROI of 100 px by 100 px is found to be sufficient. The next step is to find the ultrasound image at the seed deposition depth captured in Phase 1, which we will call I_{P1}. This image contains the previously deposited seeds as well as background noise from the phantom tissue. In order to remove the noise and other seeds from the ROI in I_{P3} the exact same ROI is taken from I_{P1} and the background is removed through a subtraction, such that a cleaner image, denoted I_{C}, is created, where I_{C}=I_{P3}−I_{P1}|. The image I_{C} is then enhanced through the same contrast stretching method given in [36], see FIG. 15B.

With the background noise and previous seeds removed from the image, the target seed is now quite distinct from the background and so the final step is the seed segmentation. A straightforward binary threshold, determined empirically to count any pixel with an intensity above 150 (on a scale from 0 to 255). As a final segmentation step all 4-connected component objects in the binary image are found and the object with the largest number of pixels is chosen as the seed. The seed location is then determined by taking the x and y centroids of all of the pixels in the seed's 4-connected object. In the following sections, the calibration of the needle steering controller is presented, followed by the needle steering and seed implant results.

### i) Model Identification

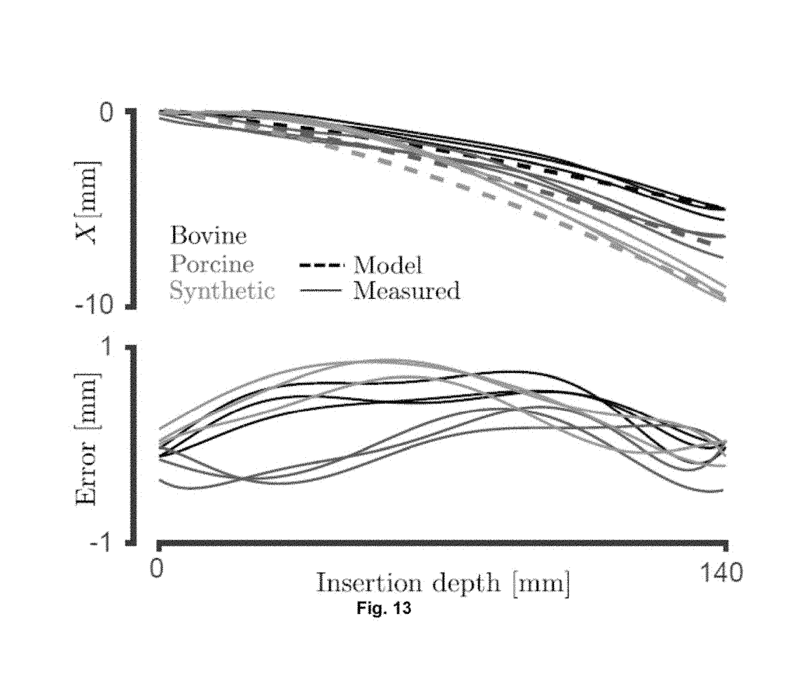

The first step in performing assisted needle steering for accurate seed deposition is to calibrate the needle steering controller. To this end, 3 needle insertions followed by withdrawals are performed in each tissue at an average velocity of 2 mm·s⁻¹. The controller is turned off and the needle insertion/withdrawal force is recorded. For verification purposes, the ultrasound probe is following the needle tip. However, in a clinical scenario the ultrasound probe could instead be maintained stationary at the maximal insertion depth to measure the needle deflection at a single depth. Following the procedure, the force applied at the needle tip is identified. The obtained force is input to the needle-tissue interaction model [33] and the needle deflection is estimated for various candidate tissue stiffness values. The optimal needle-tissue stiffness is the one that minimizes the difference between the predicted and observed needle tip deflection at the maximal insertion depth. FIG. 13 presents the results obtained with the identified model parameters. The prediction error is less than 1 mm for all tissue samples. The results, including the optimal tissue stiffness, are summarized in Table IV.

TABLE IV
Identified needle tip force (N), tissue stiffness (N ·
mm⁻²), and average absolute prediction error (mm).

| | Porcine | Bovine | Synthetic |
|---|---|---|---|
| | tissue | tissue | tissue |
| Force | 1.10 ± 0.07 | 1.26 ± 0.05 | 0.78 ± 0.12 |
| Stiffness | 72.6 | 86.5 | 36.6 |
| Mean error | 0.53 ± 0.28 | 0.83 ± 0.44 | 0.89 ± 0.62 |

ii) Seed Implant with Non-Image Based Needle Steering

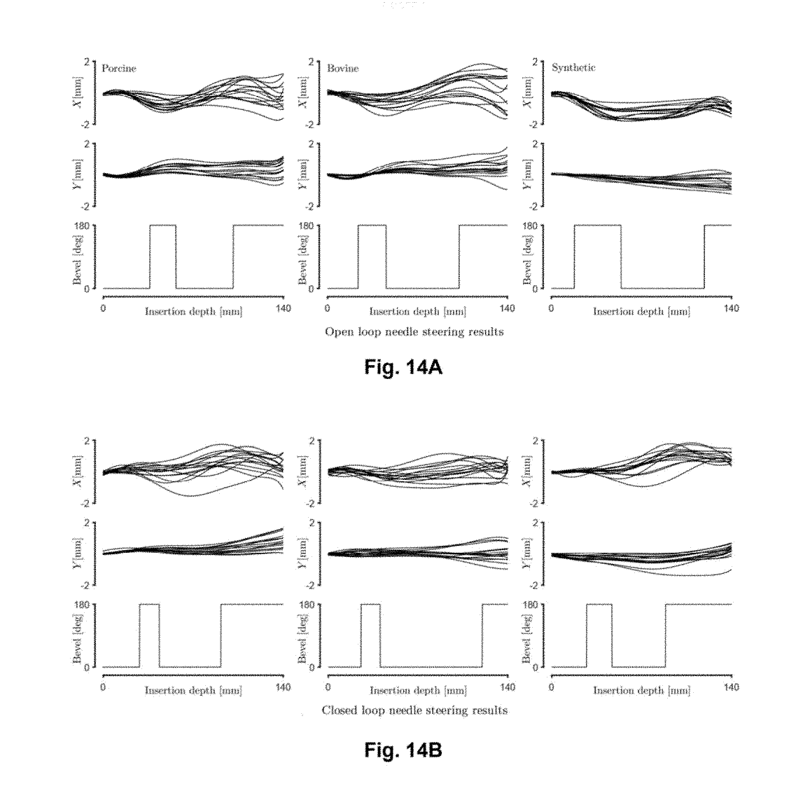

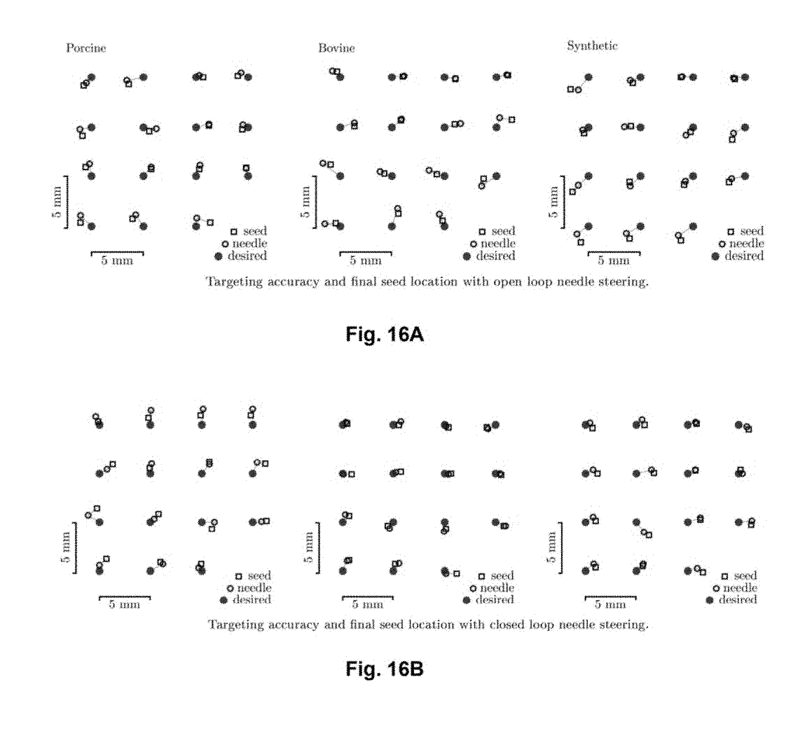

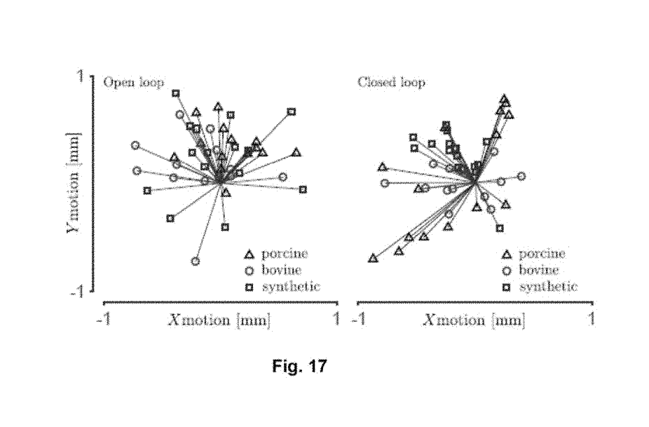

Knowing all the parameters necessary for estimating the needle tip trajectory, the depths of rotation are determined by the controller. Let us first assume that no image feedback is available. Therefore, the controller is only used prior to the needle insertion. The needle is inserted through the grid template at different locations spaced 5 mm apart as in current clinical brachytherapy. 15 insertions are performed followed by seed deposition. The path followed by the needle tip is shown in FIG. 14A along with the orientation of the needle bevel angle. Over 45 insertions, the average needle targeting accuracy in the X and Y directions is 0.93 and 0.62 mm with the highest error occurring in bovine tissue and the lowest error observed in porcine tissue. Once the needle reaches the depth of 140 mm, the seed loaded in the needle shaft is deposited in tissue and the needle is withdrawn. The final seed location with respect to the desired hypothetical seed distribution is shown in FIG. 16A. The gray solid dot indicates the desired seed location, which is defined as a point in a 2D plane parallel to the grid template at a depth of 140 mm. The final needle tip location is shown by the blue circle and the square is centroid of each seed after needle withdrawal. The average seed targeting accuracy in the X and Y planes is 0.89 and 0.60 mm, respectively. During needle withdrawal the tissue deforms and moves the seeds by up to 0.30 mm see (FIG. 17). These results are summarized in Table V.

TABLE V
Average absolute needle targeting accuracy, seed placement
error and seed deviation after needle withdrawal, and
average depth of needle rotation. Units in mm.

| | | Porcine | Bovine | Synthetic | |
|---|---|---|---|---|---|
| | | tissue | tissue | tissue | Average |
| non- | X needle | 0.69 ± 0.45 | 1.07 ± 0.41 | 1.05 ± 0.28 | 0.93 |
| image | Y needle | 0.63 ± 0.38 | 0.68 ± 0.48 | 0.56 ± 0.38 | 0.62 |
| based | X seed | 0.81 ± 0.36 | 0.86 ± 0.38 | 1.01 ± 0.46 | 0.89 |
| | Y seed | 0.53 ± 0.30 | 0.46 ± 0.37 | 0.81 ± 0.53 | 0.60 |
| | X motion | 0.27 ± 0.34 | 0.40 ± 0.37 | 0.20 ± 0.21 | 0.32 |
| | Y motion | 0.35 ± 0.17 | 0.22 ± 0.23 | 0.36 ± 0.23 | 0.31 |
| | Rotation 1 | 31.1 | 18.7 | 12.8 | |
| | Rotation 2 | 51.3 | 40.5 | 49.1 | |
| | Rotation 3 | 100.9 | 102.5 | 118.9 | |
| image | X needle | 0.51 ± 0.44 | 0.39 ± 0.26 | 0.81 ± 0.30 | 0.57 |
| based | Y needle | 0.79 ± 0.52 | 0.41 ± 0.34 | 0.40 ± 0.25 | 0.53 |
| | X seed | 0.60 ± 0.48 | 0.59 ± 0.25 | 0.21 ± 0.89 | 0.46 |
| | Y seed | 0.84 ± 0.34 | 0.34 ± 0.29 | 0.31 ± 0.31 | 0.49 |
| | X motion | 0.38 ± 0.24 | 0.31 ± 0.26 | 0.21 ± 0.21 | 0.30 |
| | Y motion | 0.47 ± 0.22 | 0.11 ± 0.09 | 0.31 ± 0.31 | 0.29 |
| | Rotation 1 | 39.2 ± 12.4 | 36.8 ± 9.3 | 38.2 ± 7.7 | |
| | Rotation 2 | 52.4 ± 13.7 | 49.6 ± 11.9 | 55.2 ± 10.3 | |
| | Rotation 3 | 98.5 ± 16.4 | 122 ± 15.8 | 95.8 ± 12.2 | |

iii) Seed Implant with Image-Based Needle Steering

Let us now assume that the position of the needle tip can be measured at any time during insertion from ultrasound images. As a result, the steering controller can update the optimal rotation depths on-line. This is expected to result in an immediate improvement of targeting accuracy since the controller replans the path towards the target given the current position of the needle tip X₀, and the number n of axial rotations that have been performed. The path followed by the needle tip is presented in FIG. 14B. The third panel shows the average position of the bevel angle. The absolute needle targeting accuracy in the X and Y planes is 0.57 and 0.53 mm, respectively. Considering the deflection along X, this corresponds to an improvement of 40% compared to the case without image feedback. The final needle tip location at the target depth and the final location of the deposited seeds are shown in FIG. 16B. The average deviation from the actual to the desired seed location is 0.46 and 0.49 mm in the vertical and horizontal planes, respectively. The second part of Table V summarizes these results.

iv. Discussion

Two different approaches have been proposed to steer a seed-carrying needle towards a pre-defined target. In the first approach the needle steering apparatus rotates the needle base at optimal depths determined preoperatively. In the second case, the the current position of the needle tip is used to update the optimal rotation depths intraoperatively.

The first method is compatible with a clinical setting where real-time measurement of the needle tip cannot be obtained during insertion. To address this limitation the steering apparatus is equipped with a force sensor that measures the needle insertion and withdrawal forces and estimates the required model parameters using the deflection measured at a single depth after insertion. 15 seeds are implanted 5 mm apart in the tissue to form a hypothetical seed distribution. The average needle and seed targeting accuracy in the controlled deflection direction is 0.93 and 0.89 mm on average, respectively.

The second method uses ultrasound images to measure the needle tip deflection in tissue as it is inserted. The controller running at 1 Hz recalculates the steering maneuvers online, such that deviations from the offline predicted path can be corrected. With this approach, the average seed placement error is reduced to 0.46 mm. Some commercially available ultrasound systems can be employed to follow the needle tip during insertion. Examples include the TargetScan from Envisioneering Medical, Overland, USA, where the 2D axial imaging plane translates within a stationary transrectal probe, and the 3D-2052 ultrasound probe from B&K Ultrasound. Peabody, USA, where the imaging plane translates axially by 70 mm. As an alternative, the Sonalis Ultrasound System from Best Medical, Pittsburgh, USA, has a longitudinal array that provides for 140 mm length of view, encompassing the bladder, the prostate and the perineum. Hence, the needle can be observed during throughout the insertion as long as it does not deflect out of the imaging plane.

Standards for seed implant quality are typically defined in terms of quantitative X-ray Computed Tomography-based postoperative dosimetric evaluation. Currently, ultrasound-based postoperative seed identification cannot be done routinely with any better than 80% accuracy [37, 38]. CT-based dosimetry evaluation requires a separate imaging session to scan the patient prostate in order to determine the final location of the seeds. This assessment is subject to anatomical variations of the prostate position and postoperative edema of the prostate gland. With the described method, assessment and corrections regarding seed implantation errors can be taken during the procedure without the need for postoperative imaging.

In summary, we demonstrate the feasibility of a new framework for accurate radioactive seed implantation and tracking during low dose rate prostate brachytherapy for prostate cancer. A hand-held needle steering apparatus controls the deflection of a seed-carrying needle during insertion such that the needle tip reaches the desired target with minimum deflection. The steering controller evaluates the effects of axial needle rotations at different depths on the needle targeting accuracy via a needle-tissue interaction model. Optimal rotation depths are determined prior to the procedure and can be updated as the needle insertion progresses. The device automatically steers the needle as the surgeon manually inserts it in tissue, keeping the surgeon in control of the procedure. Once the needle reaches the target, the surgeon can deposit the seeds in tissue as in current clinical practice. Hence, the proposed framework does not require major modifications to the operating room setup. Knowing the final needle tip location prior to seed deposition, a method is proposed to track the final seed locations after needle withdrawal, allowing the surgeon to monitor implant quality on the fly.

Despite the current clinical individual seed placement uncertainty of 5 mm, very good clinical results for brachytherapy can be achieved when the whole prostate gland is treated. This is a consequence of the large number of seeds involved in a whole gland implant (typically 80 to 100), and the addition of a 3 mm margin around the prostate to create a planning target volume to which the treatment dose is prescribed [39]. With the proposed system, the average seed placement accuracy is improved to 0.46 mm in tissue phantoms. Reducing seed placement error to this order in the clinic can enable accurate brachytherapy boost or focal treatment of dominant intra-prostatic lesions rather than treating the whole prostate gland. Seeds carrying higher radiation doses can be considered to reduce the number of implanted seeds and the targeted areas within the prostate.

Combined with improved imaging techniques [40], it is possible to identify men with low- to intermediate-risk prostate cancer who have low volume focal disease and who may be suitable for local therapy. This would result in fewer side effects to the patient including reduced urinary problems, rectal symptoms, and improved erectile function [41]. In addition, the possibility of post-treatment after focal brachytherapy is expected to be easier than after conventional treatment of the whole prostate gland. Among the options for such treatment, it is possible to treat remaining regions of the prostate volume with specific techniques of external irradiation or salvage surgery [42].

The present invention has been described above and shown in the drawings by way of exemplary embodiments and uses, having regard to the accompanying drawings. The exemplary embodiments and uses are intended to be illustrative of the present invention. It is not necessary for a particular feature of a particular embodiment to be used exclusively with that particular exemplary embodiment. Instead, any of the features described above and/or depicted in the drawings can be combined with any of the exemplary embodiments, in addition to or in substitution for any of the other features of those exemplary embodiments. One exemplary embodiment's features are not mutually exclusive to another exemplary embodiment's features. Instead, the scope of this disclosure encompasses any combination of any of the features. Further, it is not necessary for all features of an exemplary embodiment to be used. Instead, any of the features described above can be used, without any other particular feature or features also being used. Accordingly, various changes and modifications can be made to the exemplary embodiments and uses without departing from the scope of the invention as defined in the claims that follow.

### REFERENCES

All publications mentioned are incorporated herein by reference (where permitted) to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.
[1] (2015) Canadian Cancer society. Canadian cancer society's steering committee: Canadian cancer statistics 2015. [Online]. Available: http://www.cancer.ca/statistics.
[2] (2015) American cancer society. prostate cancer statistics. [Online]. Available: http://www.cancer.org/cancer/prostatecancer.
[3] M. Keyes, J. Crook, W. J. Morris, G. Morton, T. Pickles, N. Usmani, and E. Vigneault, “Canadian prostate brachytherapy in 2012,” Canadian Urological Association Journal, vol. 7, no. 1-2, p. 51, 2013.
[4] R. Taschereau, J. Pouliot, J. Roy, and D. Tremblay, “Seed misplacement and stabilizing needles in transperineal permanent prostate implants,” Radiotherapy and Oncology, vol. 55, no. 1, pp. 59-63, 2000.
[5] A. Pollack, G. K. Zagars, L. G. Smith, J. J. Lee, A. C. von Eschenbach, J. A. Antolak, G. Starkschall, and I. Rosen, “Preliminary results of a randomized radiotherapy dose-escalation study comparing 70 gy with 78 gy for prostate cancer,” Journal of Clinical Oncology, vol. 18, no. 23, pp. 3904-3911, 2000.
[6] D. Stoianovici, L. L. Whitcomb, J. H. Anderson, R. H. Taylor, and L. R. Kavoussi, “A modular surgical robotic system for image guided percutaneous procedures,” in Medical Image Computing and Computer-Assisted Intervention—MICCAI 1998. Springer, 1998, pp. 404-410.
[7] W. L. Smith, K. Surry, G. Mills, D. B. Downey, and A. Fenster, “Three dimensional ultrasound-guided core needle breast biopsy,” Ultrasound in medicine & biology, vol. 27, no. 8, pp. 1025-1034, 2001.
[8] J. A. Cadeddu, D. Stoianovici, R. N. Chen, R. G. Moore, and L. R. Kavooussi, “Stereotactic mechanical percutaneous renal access,” Journal of Endourology, vol. 12, no. 2, pp. 121-125, 1998.
[9] G. Fichtinger, T. L. DeWeese, A. Patriciu, A. Tanacs, D. Mazilu, J. H. Anderson, K. Masamune, R. H. Taylor, and D. Stoianovici, “System for robotically assisted prostate biopsy and therapy with intraoperative ct guidance,” Academic Radiology, vol. 9, no. 1, pp. 60-74, 2002.
[10] C. M. Schneider, A. M. Okamura, and G. Fichtinger, “A robotic system for transrectal needle insertion into the prostate with integrated ultrasound,” in Robotics and Automation, 2004. Proceedings. ICRA'04. 2004 IEEE International Conference on, vol. 1. IEEE, 2004, pp. 365-370.
[11] K. Cleary. M. Freedman, M. Clifford, D. Lindisch, S. Onda, and L. Jiang, “Image-guided robotic delivery system for precise placement of therapeutic agents,” Journal of Controlled release, vol. 74, no. 1, pp. 363-368, 2001.
[12] T. K. Podder, L. Beaulieu, B. Caldwell, R. A. Cormnnack, J. B. Crass, A. P. Dicker, A. Fenster, G. Fichtinger, M. A. Meltsner, M. A. Moerland et al., “(AAPM) and {GEC-ESTRO} guidelines for image-guided robotic brachytherapy: Report of task group 192,” Medical physics, vol. 41, no. 10, p. 101501, 2014.
[13] S. Okazawa, R. Ebrahimi, J. Chuang, S. Salcudean, and R. Rohling, “Hand-held steerable needle device,” Mechatronics, IEEE/ASME Transactions on, vol. 10, no. 3, pp. 285-296, June 2005.
[14] R. J. Hendrick, C. R. Mitchell, S. D. Herrell, and R. J. Webster, “Handheld transendoscopic robotic manipulators: A transurethral laser prostate surgery case study,” The International Journal of Robotics Research, p. 0278364915585397, 2015.
[15] R. J. Webster, J. S. Kim, N. J. Cowan, G. S. Chirikjian, and A. M. Okamura, “Nonholonomic modeling of needle steering,” The International Journal of Robotics Research, vol. 25, no. 5-6, pp. 509-525, 2006.
[16] S. Patil, J. Burgner, R. J. Webster, and R. Alterovitz, “Needle steering in 3-d via rapid replanning,” Robotics, IEEE Transactions on, vol. 30, no. 4, pp. 853-864, 2014.
[17] I. Khalaji, M. Hadavand, A. Asadian, R. Patel, and M. Naish, “Analysis of needle-tissue friction during vibration-assisted needle insertion,” in Intelligent Robots and Systems (IROS), 2013 IEEE/RSJ International Conference on, November 2013, pp. 4099-4104.
[18] T. Adebar, A. Fletcher, and A. Okamura, “3-d ultrasound-guided robotic needle steering in biological tissue,” Biomedical Engineering, IEEE Transactions on, vol. 61, no. 12, pp. 2899-2910, December 2014.
[19] G. Genta, Vibration dynamics and control. Springer, 2009.
[20] S. Misra, K. B. Reed, B. W. Schafer, K. Ramesh, and A. M. Okamura, “Mechanics of flexible needles robotically steered through soft tissue,” The International journal of robotics research, 2010.
[21] R. Bhat, “Natural frequencies of rectangular plates using characteristic orthogonal polynomials in rayleigh-ritz method,” Journal of Sound and Vibration, vol. 102, no. 4, pp. 493-499, 1985.
[22] S. Kirkpatrick, C. D. Gelatt, M. P. Vecchi et al., “Optimization by simulated annealing,” science, vol. 220, no. 4598, pp. 671-680, 1983.
[23] M. Waine, C. Rossa, R. Sloboda, N. Usmani, and M. Tavakoli, “3d shape visualization of curved needles in tissue from 2d ultrasound images using ransac,” in Robotics and Automation (ICRA), 2015 IEEE International Conference on, May 2015, pp. 4723-4728.
[24] C. Rossa, R. Sloboda, N. Usmani, and M. Tavakoli, “Estimating needle tip deflection in biological tissue from a single transverse ultrasound image: application to brachytherapy,” International Journal of Computer Assisted Radiology and Surgery, pp. 1-13, 2015. [Online]. Available: http://dx.doi.org/10.1007/s1548-015-1329-4
[25] G. J. Vrooijink, M. Abayazid, S. Patil, R. Alterovitz, and S. Misra, “Needle path planning and steering in a three-dimensional non-static environment using two-dimensional ultrasound images,” The International Journal of Robotics Research, pp. 1361-1374, 2014.
[26] M. Abayazid, G. J. Vrooijink, S. Patil, R. Alterovitz, and S. Misra, “Experimental evaluation of ultrasound-guided 3d needle steering in biological tissue,” International journal of computer assisted radiology and surgery, vol. 9, no. 6, pp. 931-939, 2014.
[27] J. Hong, T. Dohi, M. Hashizume, K. Konishi, and N. Hata, “An ultrasound-driven needle-insertion robot for percutaneous cholecystostomy,” Physics in Medicine and Biology, vol. 49, no. 3, p. 441, 2004.
[28] M. Lachaine and T. Falco, “Intrafractional prostate motion management with the clarity autoscan system,” Med. Phys. Int, vol. 1, no. 1, pp. 72-80, 2013.
[29] J. Schlosser, K. Salisbury, and D. Hristov, “Tissue displacement monitoring for prostate and liver igrt using a robotically controlled ultrasound system,” Medical Physics, vol. 38, no. 6, pp. 3812-3812, 2011.
[30] M. Abayazid, R. Roesthuis, R. Reilink, and S. Misra, “Integrating deflection models and image feedback for real-time flexible needle steering,” Robotics, IEEE Transactions on, vol. 29, no. 2, pp. 542-553, April 2013.
[31] D. C. Rucker, J. Das, H. B. Gilbert, P. J. Swaney, M. Miga, N. Sarkar, R. J. Webster et al., “Sliding mode control of steerable needles,” Robotics, IEEE Transactions on, vol. 29, no. 5, pp. 1289-1299, 2013.
[32] M. Keyes. D. Schellenberg, V. Moravan, M. McKenzie, A. Agranovich, T. Pickles, J. Wu, M. Liu, J. Bucci, and W. J. Morris, “Decline in urinary retention incidence in 805 patients after prostate brachytherapy: The effect of learning curve?” International Journal of Radiation Oncology* Biology* Physics, vol. 64, no. 3, pp. 825-834, 2006.
[33] C. Rossa, M. Khadem, R. Sloboda, N. Usmani, and M. Tavakoli. “Adaptive quasi-static modeling of needle deflection during steering in soft tissue.” IEEE Robotics and Automation Letters 1(2), 916-923, 2016.
[34] S. M. LaValle and J. J. Kuffner. “Randomized kino-dynamic planning. The International Journal of Robotics Research 20(5), 378-400, 2001.
[35] S. Patil et al. “Needle steering in 3D via rapid replanning.” IEEE Transactions on Robotics 30(4), 853-864, 2014.
[36] M. Waine, C. Rossa, R. Sloboda, N. Usmani, and M. Tavakoli. “Needle tracking and deflection prediction for robot-assisted needle insertion using 2D ultrasound images.” Journal of Medical Robotics Research 01(01), 1640001, 2016.
[37] B. Han, K. Wallner, G. Merrick, W. Butler, S. Sutlief, and J. Sylvester. “Prostate brachytherapy seed identification on post-implant trus images.” Medical Physics 30(5), 898-900, 2003.
[38] Z. Wei, L. Gardi, D. Downey, and A. Fenster. “Automated localization of implanted seeds in 3d trus images used for prostate brachytherapy.” Medical Physics 33(7), 2404-2417, 2006.
[39] C. Salembier, P. Lavagnini, P. Nickers, P. Mangili, A. Rijnders, A. Polo et al. “Tumour and target volumes in permanent prostate brachytherapy: a supplement to the estro/eau/eortc recommendations on prostate brachytherapy. Radiotherapy and oncology 83(1), 3-10, 2007.
[40] M. Atri, M. Gertner, M. Haider, R. Weersink, and J. Trachtenberg. “Contrast-enhanced ultrasonography for real-time monitoring of interstitial laser therapy in the focal treatment of prostate cancer.” Canadian Urological Association Journal 3(2), 125-30, 2009.
[41] S. Langley, U. H. Ahmed, B. Al-Qaisieh, D. Bostwick, L. Dickinson, and F. Veiga et al. “Report of a consensus meeting on focal low dose rate brachytherapy for prostate cancer.” BJU International 109(s1), 7-16, 2012.
[42] J. M. Cosset, X. Cathelineau, G. Wakil, N. Pierrat, O. Quenzer, and D. Prapotnich et al. “Focal brachytherapy for selected low-risk prostate cancers: a pilot study.” Brachytherapy 12(4), 331-337, 2013.

The embodiments of the invention in which an exclusive property or privilege is claimed are defined as follows:

1. A hand-held device for assisted steering of a percutaneously inserted needle comprising:
(a) a handle for manual gripping of the device by a user of the device;
(b) an actuation unit attached to the handle, the actuation unit comprising:
(i) a rotary actuator for axially rotating the needle relative to the handle;
(ii) an axial actuator for inducing axial micro-vibrations of the needle relative to the handle;
wherein the rotary actuator and the axial actuator are simultaneously operable to simultaneously axial rotate the needle relative to the handle and induce axial micro-vibrations of the needle relative to the handle;
(c) a haptic feedback unit for inducing vibrations in the handle.

2. The device of claim 1 further comprising a sensor unit comprising at least one sensor attached to the handle for generating, in response to movement of the device, an electronic signal indicative of a needle insertion parameter comprising one or a combination of the needle position, a needle orientation, a needle axial rotation angle, a needle velocity, and a needle acceleration.

3. The device of claim 2 wherein the at least one sensor comprises one or a combination of an accelerometer or a gyroscopic sensor.

4. A computer-implemented system for assisted steering of a percutaneously inserted needle comprising:
(a) a hand-held device comprising;
(i) a handle for manual gripping of the device by a user of the device;
(ii) an actuation unit attached to the handle, the actuation unit comprising:
(1) a rotary actuator for axially rotating the needle relative to the handle;
(2) an axial actuator for inducing axial micro-vibrations of the needle relative to the handle,
wherein the rotary actuator and the axial actuator are simultaneously operable to simultaneously axial rotate the needle relative to the handle and induce axial micro-vibrations of the needle relative to the handle;
(iii) a haptic feedback unit for inducing vibrations in the handle;
(b) a sensor unit comprising at least one sensor for generating, in response to movement of the device, an electronic signal indicative of a needle insertion parameter comprising one or a combination of the needle position, a needle orientation, a needle axial rotation angle, a needle velocity, and a needle acceleration;
(c) a display device; and
(d) a computer operatively connected to the device and the display device, the computer comprising a processor and a memory comprising a non-transitory computer readable medium storing instructions executable by the processor to implement, in real-time with the insertion of the needle, a method comprising the steps of:
(i) determining a location of a portion of the needle;
(ii) calculating a needle insertion parameter comprising one or a combination of a needle position, a needle orientation, a needle axial rotation angle, a needle velocity, and a needle acceleration, wherein the calculating is based on an electronic signal from the sensor unit;
(iii) calculating a needle shape and a needle position, wherein the calculating is based on one or a combination of the determined location of the portion of the needle and the calculated needle insertion parameter,
(iv) displaying on the display device one or a combination of the calculated needle shape, the calculated needle position, and the calculated needle insertion parameter,
(v) calculating a correction to the needle insertion parameter for a target needle trajectory, wherein the calculating is based on one or a combination of the calculated needle shape, the calculated needle position, and the calculated needle insertion parameter, and wherein the correction to needle insertion parameter comprises at least either a correction to the needle axial rotation angle or a needle rotation depth paired with a discrete needle rotation angle;
(vi) controlling the rotary actuator of the device to axially rotate the needle by either the correction to the needle axial rotation angle or by the discrete needle rotation angle at the paired needle rotation depth;
(vii) activating the haptic feedback unit of the device to vibrate the handle of the device in a vibration pattern, wherein the vibration pattern is determined by a rules database depending on one or a combination of the calculated needle shape, the calculated needle position, and the calculated correction to the needle insertion parameter; and
(viii) repeating steps (i) to (vi).

5. The system of claim 4 wherein the sensor of the sensor unit is attached to the handle and comprises one or a combination of an accelerometer and a gyroscopic sensor,

6. The system of claim 4 wherein the sensor unit comprises a camera for tracking the position of the device.

7. The system of claim 4 wherein the step of determining the location of the portion of the needle comprises processing an ultrasound image of the needle.
